(19) United States
(12) Patent Application Publication
(10) Pub. No.: US 2021/0208098 A1
(43) Pub. Date: Jul. 8, 2021

(54) LABEL-FREE NANOSENSORS FOR DETECTION OF GLYCOPROTEINS

(71) Applicants: Seyyed Alireza Hashemi, Frankfurt (DE); Sonia Bahrani, Frankfurt (DE); Seyyed Mojtaba Mousavi, Frankfurt (DE); Seyyed Hadi Ahmadi, Frankfurt (DE); Mohammad Firoozsani, Frankfurt (DE)

(72) Inventors: Seyyed Alireza Hashemi, Frankfurt (DE); Sonia Bahrani, Frankfurt (DE); Seyyed Mojtaba Mousavi, Frankfurt (DE); Seyyed Hadi Ahmadi, Frankfurt (DE); Mohammad Firoozsani, Frankfurt (DE)

(73) Assignees: Seyyed Alireza Hashemi, Frankfurt (DE); Seyyed Hadi Ahmadi, Frankfurt (DE); Mohammad Firoozsani, Frankfurt (DE)

(21) Appl. No.: 17/205,794

(22) Filed: Mar. 18, 2021

Related U.S. Application Data
(60) Provisional application No. 63/010,991, filed on Apr. 16, 2020.

(51) Int. Cl. G01N 27/411

(52) U.S. Cl. CPC G01N 27/4115

(57) ABSTRACT

A method for detecting glycoproteins in aqueous samples. The method includes putting an aqueous sample in contact with a diagnostic kit, obtaining an electrochemical pattern of the aqueous sample by applying an electrical potential to the diagnostic kit, and detecting a glycoprotein status of the aqueous sample based on the presence of a peak in the electrochemical pattern of the aqueous sample. The diagnostic kit includes a counter electrode, a reference electrode, and a working electrode including a label-free nanosensor deposited on a substrate. The label-free nanosensor includes a modified graphene oxide (GO) sheet and a signal amplifying agent loaded onto the modified GO sheet. The modified GO sheet includes a modifying agent conjugated to a GO sheet. The modifying agent includes 1-ethyl-3-(3-dimethylaminopropyl) carbodiimide (EDC), N-hydroxysuccinimide (NHS), 8-hydroxyquinoline (8H), and hydroxylammonium chloride. The signal amplifying agent includes at least one of an amine-functionalized gold nanoparticle and a silver nanoparticle.

### CROSS-REFERENCE TO RELATED APPLICATION

This application claims the benefit of priority from pending U.S. Provisional Patent Application Ser. No. 63/010,991, filed on Apr. 16, 2020, and entitled “RAPID LABEL-FREE ELECTROCHEMICAL BIOSENSOR FOR DETECTION OF GLYCOPROTEINS,” which is incorporated herein by reference in its entirety.

### TECHNICAL FIELD

The present disclosure generally relates to biosensors, particularly to electrochemical biosensors to detect glycoproteins, and more particularly to label-free electrochemical biosensors for detecting viral glycoproteins.

### BACKGROUND

Glycoproteins play an essential role in various biological processes of living organisms, such as protein folding, cell signaling, cell proliferation, and cell-cell interaction. Recent studies have also demonstrated presence of viral or bacterial surface glycoproteins in the process of most infections and immune responses. As a result, quantitation and identification of glycoproteins may be used as an essential biomarker for early detection of pathologies processes, while its increasing content within biological samples may be used as a promising biological marker.

Conventionally, various techniques have been developed to identify and quantify glycoproteins within aquatic biological matters, including enzyme-linked immune-sorbent assay (ELISA), capillary electrophoresis high-performance anion exchange chromatography, and liquid chromatography. Although conventional techniques provide some advantages for detection of target glycoproteins, a majority of them suffer from expensive cost, complex specimen pretreatment, time-consuming processes, a requirement for skilled personnel, poor physical or chemical stability, and complicated processes for obtaining biological reagents, such as antibodies, DNA, antigens, and cells which restrict their applicability.

Electrochemical biosensors have superior properties over other existing measurement systems due to providing rapid, simple, and low-cost on-field detection. Moreover, electrochemical measurement protocols are suitable for mass fabrication of miniaturized devices. Electrochemical biosensors have played a significant role in the move towards simplified testing for point-of-care usage. Also, label-free electrochemical biosensors have shed new light on bio-analysis due to their low cost, multiplexed detection capabilities, and miniaturization ease without any other biochemical processes.

Hence, there is a need for label-free, simple, cost-effective, sensitive, stable, and time-saving biosensors capable of detecting a wide variety of glycoproteins. Also, there is a need for a rapid, practical, and reliable diagnostic assay based on label-free electrochemical biosensors for tracing and quantifying glycoproteins in biological samples without any need for using biological reagents like antibodies.

### SUMMARY

This summary is intended to provide an overview of the subject matter of the present disclosure and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary diagnostic kit for detecting glycoproteins in aqueous samples. In an exemplary embodiment, exemplary diagnostic kit may include a working electrode, a reference electrode, and a counter electrode. In an exemplary embodiment, the working electrode may include an exemplary label-free nanosensor deposited on a substrate. In an exemplary embodiment, exemplary label-free nanosensor may include a modified graphene oxide (GO) sheet and a signal amplifying agent loaded onto the modified GO sheet. In an exemplary embodiment, the modified graphene oxide (GO) sheet may include a modifying agent conjugated to a GO sheet. In an exemplary embodiment, the modifying agent may include 1-ethyl-3-(3-dimethylaminopropyl) carbodiimide (EDC), N-hydroxysuccinimide (NHS), 8-hydroxyquinoline (8H), and hydroxylammonium chloride. In an exemplary embodiment, the signal amplifying agent may include at least one of an amine-functionalized gold nanoparticle and a silver nanoparticle.

In an exemplary embodiment, the modifying agent may include the EDC with a concentration between about 1% and about 20% by weight of the GO sheet, the NHS with a concentration between about 1% and about 20% by weight of the GO sheet, the 8H with a concentration between about 10% and about 50% by weight of the GO sheet, and the hydroxylammonium chloride with a concentration between about 10% and about 50% by weight of the GO sheet. In an exemplary embodiment, the modifying agent may further include cyclodextrin with a concentration between about 10% and about 50% by weight of the GO sheet. In an exemplary embodiment, the amine-functionalized gold nanoparticle may include at least one of an amine-functionalized gold nanostar, an amine-functionalized gold nanorod, an amine-functionalised gold nanowire, an amine-functionalized gold spherical nanoparticle, an amine-functionalized gold nanoplate, and an amine-functionalized gold cubic nanostructure.

In another general aspect, the present disclosure describes an exemplary method for detecting glycoproteins in aqueous samples. Exemplary method may include putting an aqueous sample in contact with exemplary diagnostic kit, obtaining an electrochemical pattern of the aqueous sample by applying an electrical potential to exemplary diagnostic kit, and detecting a glycoprotein status of the aqueous sample based on presence of a peak in the electrochemical pattern of the aqueous sample. In an exemplary embodiment, detecting the glycoprotein status of the aqueous sample may include detecting that a glycoprotein may be present in the aqueous sample if the electrochemical pattern may contain a peak and detecting that a glycoprotein may be absent in the aqueous sample if the electrochemical pattern may lack a peak. In an exemplary embodiment, the peak may include a current intensity and a voltage position.

In an exemplary embodiment, exemplary method may further include identifying the glycoprotein in the aqueous sample by comparing the peak of the electrochemical pattern with standard peaks of standard electrochemical patterns in a database. In an exemplary embodiment, the database may include a plurality of datasets. In an exemplary embodiment, each dataset may be associated with a standard glycoprotein. In an exemplary embodiment, each dataset may include a standard electrochemical pattern of the standard glycoprotein and a calibration curve. In an exemplary embodiment, the standard electrochemical pattern may include a standard peak, including a standard voltage position and a standard current intensity. In an exemplary embodiment, the calibration curve may relate the standard current intensity of the standard electrochemical pattern to a concentration of the standard glycoprotein.

In an exemplary embodiment, comparing the peak of the electrochemical pattern with the standard peaks of the standard electrochemical patterns in the database may include determining a type of the glycoprotein by finding a standard glycoprotein in the database and measuring a concentration of the glycoprotein based on the calibration curve of the standard glycoprotein. In an exemplary embodiment, finding the standard glycoprotein in the database may include comparing the voltage position of the peak with standard voltage positions of the standard peaks in the database.

In an exemplary embodiment, exemplary method may further include generating a database. In an exemplary embodiment, generating the database may include obtaining a plurality of standard electrochemical patterns of a plurality of standard glycoproteins and plotting a calibration curve for each standard glycoprotein. In an exemplary embodiment, each standard electrochemical pattern of the standard glycoprotein may include a standard peak, including a standard voltage position and a standard current intensity. In an exemplary embodiment, plotting a calibration curve for each standard glycoprotein may include relating the standard current intensity of each standard electrochemical pattern to a concentration of the standard glycoprotein.

In an exemplary embodiment, applying the electrical potential to the diagnostic kit may include applying a predetermined electrical potential between about −1 V and about 1 V to the diagnostic kit. In an exemplary embodiment, applying the electrical potential to the diagnostic kit may include applying a predetermined electrical potential to the diagnostic kit through an electrochemical system connected to the diagnostic kit. In an exemplary embodiment, obtaining the electrochemical pattern of the aqueous sample may include obtaining at least one of a cyclic voltammetry (CV) pattern, a differential pulse voltammetry (DPV) pattern, an electrochemical impedance spectroscopy (EIS) pattern, a square wave voltammetry (SWV) pattern, and a pattern of an amperometry assay of the aqueous sample.

In an exemplary embodiment, detecting glycoproteins in the aqueous samples may include detecting at least one of viral glycoproteins, collagens, and antibodies in the aqueous samples. In an exemplary embodiment, detecting the viral glycoproteins may include detecting at least one of coronaviruses, influenza viruses, and Newcastle disease viruses. In an exemplary embodiment, putting the aqueous sample in contact with exemplary diagnostic kit may include putting at least one of a serum sample, a urine sample, a cerebrospinal fluid sample, a saliva sample, a blood sample, a mucus sample, a swab sample, and a buffer sample in contact with exemplary diagnostic kit.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A illustrates an exemplary method for detecting glycoproteins in aqueous samples, consistent with one or more exemplary embodiments of the present disclosure.

FIG. 1B illustrates an exemplary implementation of exemplary method for detecting glycoproteins in aqueous samples, consistent with one or more exemplary embodiments of the present disclosure.

FIG. 1C illustrates an exemplary method for identifying the glycoprotein in the aqueous sample by comparing a peak of an electrochemical pattern with standard peaks of standard electrochemical patterns in a database, consistent with one or more exemplary embodiments of the present disclosure.

FIG. 1D illustrates another exemplary implementation of exemplary method for detecting glycoproteins in aqueous samples, consistent with one or more exemplary embodiments of the present disclosure.

FIG. 1E illustrates an exemplary method for generating a database including a plurality of datasets of a plurality of standard glycoproteins, consistent with one or more exemplary embodiments of the present disclosure.

FIG. 2A illustrates a schematic of an exemplary label-free nanosensor configured to detect glycoproteins in aqueous samples, consistent with one or more exemplary embodiments of the present disclosure.

FIG. 2B illustrates a schematic of putting an aqueous sample in contact with exemplary diagnostic kit, consistent with one or more exemplary embodiments of the present disclosure.

FIG. 3 illustrates a schematic of an exemplary electrochemical system for detecting glycoproteins in aqueous samples, consistent with one or more exemplary embodiments of the present disclosure,

FIG. 4 illustrates an exemplary computer system in which an embodiment of the present disclosure, or portions thereof, may be implemented as computer-readable code, consistent with one or more exemplary embodiments of the present disclosure.

FIG. 5A illustrates a Fourier-transform infrared (FTIR) spectrum of graphene oxide (GO) sheets, consistent with one or more embodiments of the present invention.

FIG. 5B illustrates an FTIR spectrum of modified GO sheets, including GO sheets modified with 1-ethyl-3-(3-dimethylaminopropyl) carbodiimide (EDC), N-hydroxysuccinimide (NHS), 8-hydroxyquinoline (8H), and hydroxylammonium chloride, consistent with one or more embodiments of the present invention.

FIG. 5C illustrates an FTIR spectrum of gold nanostars (Au NS), consistent with one or more embodiments of the present disclosure.

FIG. 6A illustrates a transmission electron microscopy (TEM) image of GO sheets, consistent with one or more embodiments of the present disclosure.

FIG. 6B illustrates a TEM image of modified GO sheets, including GO sheets modified with EDC. NHS, 8H, and hydroxylammonium chloride, consistent with one or more embodiments of the present disclosure.

FIG. 6C illustrates TEM images of the gold nanostars (Au NS) consistent with one or more embodiments of the present disclosure.

FIG. 7 illustrates a result of cyclic voltammetry (CV) analysis of glassy carbon electrode (GCE) as an unmodified working electrode, and GCE deposited with modified GO sheets (GCE-GO-8H-EDC-NHS), GCE deposited with Au NSs (GCE-AuNS), and exemplary label-free nanosensor (GCE-GO-8H-EDC-NHS-AuNS), consistent with one or more embodiments of the present disclosure.

FIG. 8 illustrates a result of electrochemical impedance spectroscopy (EIS) analysis of glassy carbon electrode (GCE) as an unmodified working electrode, and GCE deposited with modified GO sheets (GCE-GO-8H-EDC-NHS). GCE deposited with Au NSs (GCE-AuNS), and exemplary label-free nanosensor (GCE-GO-8H-EDC-NHS-AuNS), consistent with one or more embodiments of the present disclosure.

FIG. 9A illustrates a differential pulse voltammetry (DPV) pattern of whole glycoproteins of infectious bronchitis virus (IBV), consistent with one or more embodiments of the present disclosure,

FIG. 9B illustrates a DPV pattern of spike glycoprotein of IBV in phosphate-buffered solution (PBS) consistent with one or more embodiments of the present disclosure.

FIG. 9C illustrates a calibration curve of IBV in PBS, consistent with one or more embodiments of the present disclosure.

FIG. 9D illustrates a DPV pattern of spike glycoprotein of IBV in a human blood plasma sample, consistent with one more embodiments of the present disclosure.

FIG. 9E illustrates a calibration curve of IBV in a human blood plasma sample, consistent with one or more embodiments of the present disclosure.

FIG. 9F illustrates DPV patterns of IBV in oropharyngeal swabs of chickens infected with wild type IBV, consistent with one or more embodiments of the present disclosure.

FIG. 9G illustrates a DPV pattern of BV in a tracheal mucosa layer extracted from an infected bird with a wild-type strain of IBV, consistent with one or more embodiments of the present disclosure.

FIG. 9H illustrates DPV patterns of IBV in extracted blood samples from infected chickens with the wild-type strain of IBV, consistent with one or more embodiments of the present disclosure.

FIG. 9I illustrates the effect of electroactive interfaces on the DPV pattern obtained using exemplary diagnostic kit, consistent with one or more embodiments of the present disclosure.

FIG. 10A illustrates a DPV pattern of severe acute respiratory syndrome coronavirus-2 (SARS-CoV-2) in phosphate-buffered solution (PBS), consistent with one or more embodiments of the present disclosure.

FIG. 10B illustrates a calibration curve of SARS-CoV-2 in PBS, consistent with one or more embodiments of the present disclosure.

FIG. 10C illustrates a DPV pattern of SARS-CoV-2 in blood samples of infected people, consistent with one or more embodiments of the present disclosure.

FIG. 10D illustrates a DPV pattern of SARS-CoV-2 in a saliva sample of an infected person, consistent with one or more embodiments of the present disclosure.

FIG. 10E illustrates a DPV pattern of SARS-CoV-2 in an oropharyngeal swab sample of an infected person, consistent with one or more embodiments of the present disclosure.

FIG. 11A illustrates a DPV pattern of Newcastle disease virus (LaSota strain), consistent with one or more exemplary embodiments of the present disclosure.

FIG. 11B illustrates a DPV pattern of Newcastle disease virus (V4 strain), consistent with one or more exemplary embodiments of the present disclosure.

FIG. 12A illustrates a DPV pattern of avian influenza virus, consistent with one or more exemplary embodiments of the present disclosure.

FIG. 12B illustrates a DPV pattern of H₁N₁ strain of influenza virus, consistent with one or more exemplary embodiments of the present disclosure.

FIG. 12C illustrates a DPV pattern of HSNi strain of influenza virus, consistent with one or more exemplary embodiments of the present disclosure.

FIG. 13A illustrates a DPV pattern of human type I collagen, consistent with one or more exemplary embodiments of the present disclosure.

FIG. 13B illustrates a DPV pattern of porcine type I collagen, consistent with one or more exemplary embodiments of the present disclosure.

FIG. 14 illustrates a DPV pattern of monoclonal IgG antibody against S1 part of S spike glycoprotein of SARS-CoV-2, consistent with one or more exemplary embodiments of the present disclosure.

FIG. 15 illustrates the transmission electron microscopy (TEM) image of modified GO sheets, including GO sheets modified with EDC, NIS, 8H, hydroxylammonium chloride, and β-cyclodextrin, consistent with one or more embodiments of the present disclosure.

FIG. 16A illustrates an X-ray powder diffraction (XRD) spectrum of silver nanowires (Ag NWs), consistent with one or more embodiments of the present disclosure.

FIG. 16B illustrates a field-emission scanning electron microscopy (FESEM) image of Ag NWs, consistent with one or more exemplary embodiments of the present disclosure.

FIG. 17A illustrates a DPV pattern of GO sheets modified with EDC, NHS, 8H, hydroxylammonium chloride, and β-cyclodextrin, consistent with one or more exemplary embodiments of the present disclosure.

FIG. 17B illustrates a DPV pattern of SAR-CoV-2 glycoproteins utilizing exemplary label-free nanosensor including modified GO sheets, containing GO sheets modified with EDC, NHS, 8H, hydroxylammonium chloride, and β-cyclodextrin, along with Ag NWs as an amplifying agent, consistent with one or more embodiments of the present disclosure.

FIG. 17C illustrates a DPV pattern of SARS-CoV-2 glycoproteins obtained by utilizing exemplary label-free nanosensor containing modified GO sheets, including GO sheets modified with EDC, NHS, 8H, hydroxylammonium chloride, and β-cyclodextrin, along with Au NSs as an amplifying agent, consistent with one or more embodiments of the present disclosure.

FIG. 17D illustrates a DPV pattern of glycoproteins of SARS-CoV-2 and H₁N₁ strain of influenza virus detected utilizing an exemplary label-free nanosensor containing modified GO sheets, including GO sheets modified with EDC, NIS, 8H, hydroxylammonium chloride, and β-cyclodextrin, consistent with one or more embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well-known methods, procedures, components and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown but is to be accorded the widest possible scope consistent with the principles and features disclosed herein.

Detection of glycoproteins, which are important markers found on surfaces of various types of cells and pathogenic organisms, may have great importance because glycoproteins may closely associate with severe human diseases like cancer, rheumatoid arthritis, immunodeficiency diseases, and viral infections. Utilizing improved electrochemical sensing interfaces is crucial in such electrochemical sensors leading to accurate, sensitive, and stable glycoprotein detection. Therefore, the development of bio-electrochemical sensing interfaces that provide a label-free platform for sensitive and selective detection of glycoproteins is of great importance in medical diagnostics. The present disclosure describes an exemplary method and an exemplary diagnostic kit, including an exemplary sensitive label-free nanosensor for specific detection of glycoproteins in aqueous samples. Exemplary diagnostic kit may help diagnose diseases, including viral diseases, bacterial infections, fungal infections, cancers, immunodeficiency diseases, metabolic disorders, and glycoprotein storage diseases.

The present disclosure describes an exemplary rapid method for detecting a trace of different kinds of pathogenic animal/human glycoproteins utilizing an exemplary highly sensitive diagnostic kit. Exemplary diagnostic kit may detect glycoproteins in aqueous samples without any need for extraction or using biological markers. Exemplary diagnostic kit may include an exemplary label-free nanosensor with superior detection limit and sensitivity toward detection/quantification of glycoprotein-based structures and found to be a reliable and fast platform for detecting viral diseases in their silent stages and checking the progress of illnesses via monitoring the concentration of viruses within biological fluids.

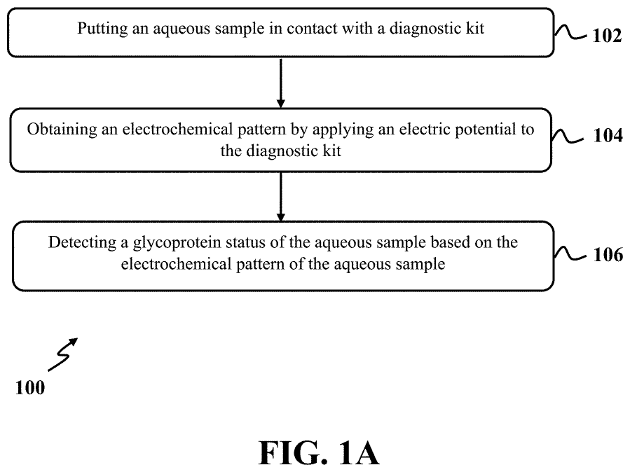

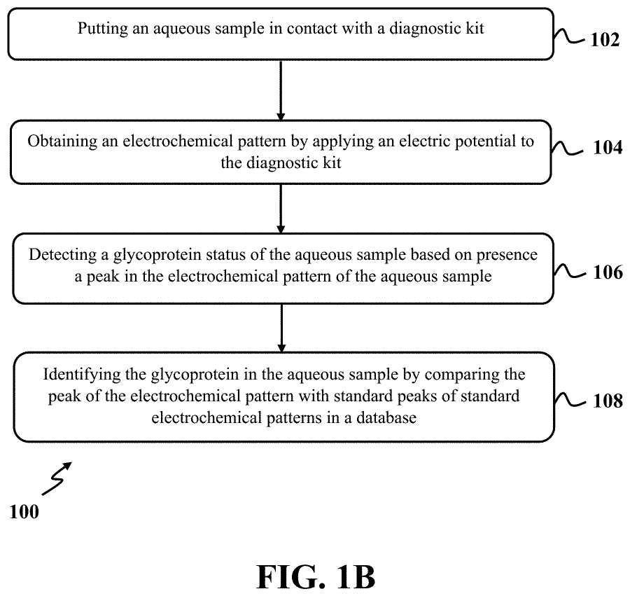

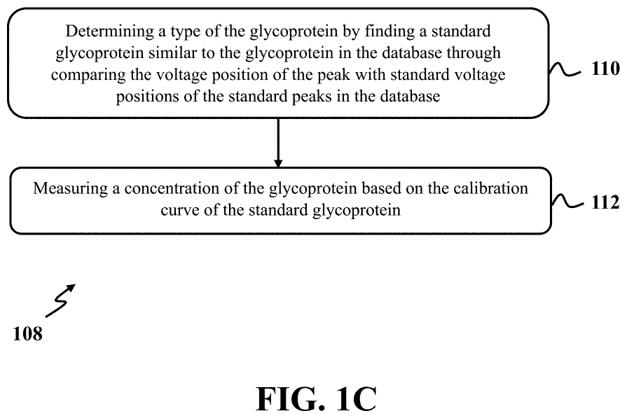

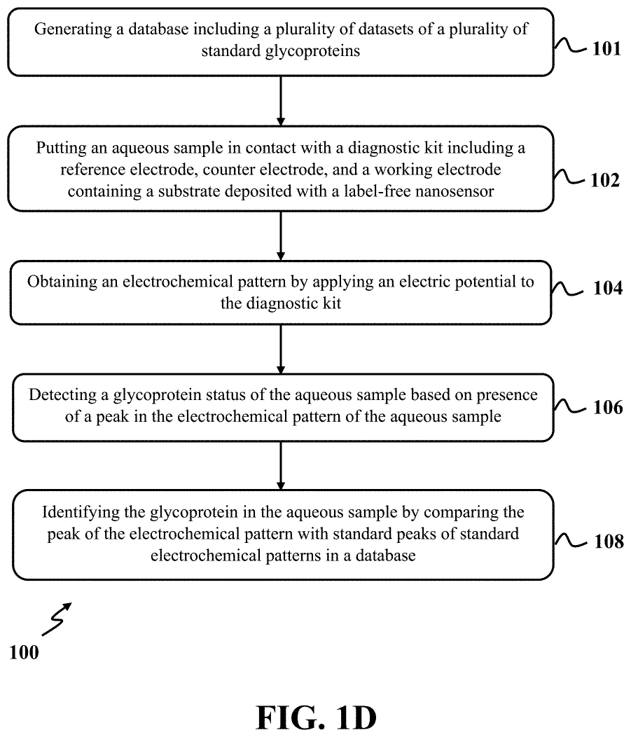

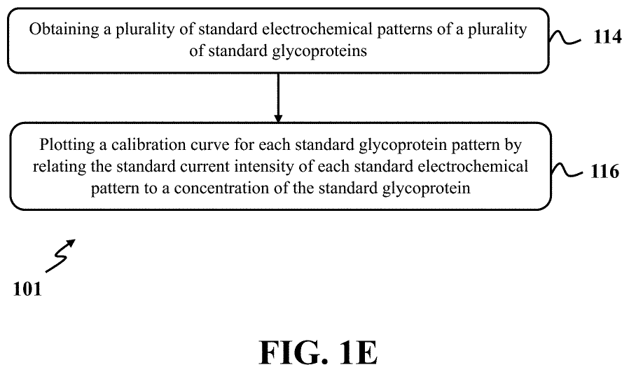

FIG. 1A illustrates an exemplary method 100 for detecting glycoproteins in aqueous samples, consistent with one or more exemplary embodiments of the present disclosure. Exemplary method 100 may include putting an aqueous sample in contact with an exemplary diagnostic kit (step 102), obtaining an electrochemical pattern by applying an electrical potential to exemplary diagnostic kit (step 104), and detecting a glycoprotein status of the aqueous sample based on the electrochemical pattern of the aqueous sample (step 106).

In an exemplary implementation, method 100 may be utilized for real-time and fast detection of glycoproteins in aqueous samples. In an exemplary implementation, method 100 may allow for quick glycoprotein detection in aqueous samples in about a minute, in an exemplary embodiment, exemplary method and exemplary diagnostic kit may be used for simultaneous detection of multiple glycoproteins in the aqueous samples. In an exemplary embodiment, simultaneous detection of multiple glycoproteins in the aqueous samples may include simultaneously determining types and concentrations of multiple glycoproteins in an aqueous sample.

In an exemplary embodiment, detecting glycoproteins in the aqueous samples may include detecting at least one of viral glycoproteins, collagens, and antibodies in the aqueous samples. In an exemplary embodiment, detecting the viral glycoproteins may include detecting at least one of coronaviruses, influenza viruses, and Newcastle disease viruses. In an exemplary embodiment, detecting glycoproteins of coronaviruses may include detecting glycoproteins of β-coronaviruses and γ-coronaviruses. In an exemplary embodiment, detecting glycoproteins of β-coronaviruses may include detecting glycoproteins of severe acute respiratory syndrome coronavirus-2 (SARS-CoV-2). In an exemplary embodiment, detecting glycoproteins of γ-coronaviruses may include detecting glycoproteins of infectious bronchitis virus (IBV).

In an exemplary embodiment, detecting glycoproteins influenza viruses may include detecting glycoproteins of at least one of H₁N₁ strain and H₃N₂ strain of avian influenza viruses. In an exemplary embodiment, detecting glycoproteins Newcastle disease viruses (NDVs) may include detecting glycoproteins of at least one of LaSota strain and V4 strain of NDVs. In an exemplary embodiment, detecting collagens may include detecting at least one of human collagen type I and porcine collagen type I. In an exemplary embodiment, detecting antibodies may include detecting a monoclonal IgG antibody of S1 part of spike (S) glycoprotein of SARS-CoV-2. In an exemplary embodiment, detecting glycoproteins in the aqueous samples may include detecting cell-membrane glycoproteins and bacterial glycoproteins in aqueous samples. In an exemplary embodiment, detecting the viral glycoproteins may include detecting whole-virus glycoproteins, viral spike glycoproteins, and portions of viral glycoproteins.

In further detail with respect to step 102, in an exemplary embodiment, putting an aqueous sample in contact with exemplary diagnostic kit may include at least one of a serum sample, a urine sample, a cerebrospinal fluid sample, a saliva sample, a blood sample, a mucus sample, a swab sample, and a buffer sample being put in contact with exemplary diagnostic kit. In an exemplary embodiment, the aqueous sample may have a pH level of about 7. In an exemplary embodiment, putting the aqueous sample in contact with exemplary diagnostic kit may include adding or dropping the aqueous sample to exemplary diagnostic kit.

In an exemplary embodiment, exemplary diagnostic kit may be configured to conduct electrochemical measurements. In an exemplary embodiment, exemplary diagnostic kit may be sterilized before putting the aqueous sample in contact with exemplary diagnostic kit. In an exemplary embodiment, the diagnostic kit may include a reference electrode, counter electrode, and a working electrode. In an exemplary embodiment, the working electrode may include exemplary label-free nanosensor deposited on a substrate.

In an exemplary embodiment, putting the aqueous sample in contact with exemplary diagnostic kit may include putting the aqueous sample in contact with the working electrode, the counter electrode, and the reference electrode. In an exemplary embodiment, the counter electrode may include at least one of a carbon electrode and a platinum electrode. In an exemplary embodiment, the reference electrode may include at least one of a silver (Ag) electrode and a silver/silver chloride (Ag/AgCl) electrode. In an exemplary embodiment, the working electrode may include an exemplary label-free nanosensor deposited on a substrate. In the present disclosure, “deposited” on the substrate may refer to coated on the substrate. In the present disclosure, “deposited” with an exemplary label-free nanosensor may refer to coated with an exemplary label-free nanosensor. In an exemplary embodiment, the substrate may include at least one of a carbon electrode, a gold electrode, and a platinum electrode. In an exemplary embodiment, the carbon electrode may include at least one of activated carbon, mesoporous carbon, graphite, and carbonaceous material.

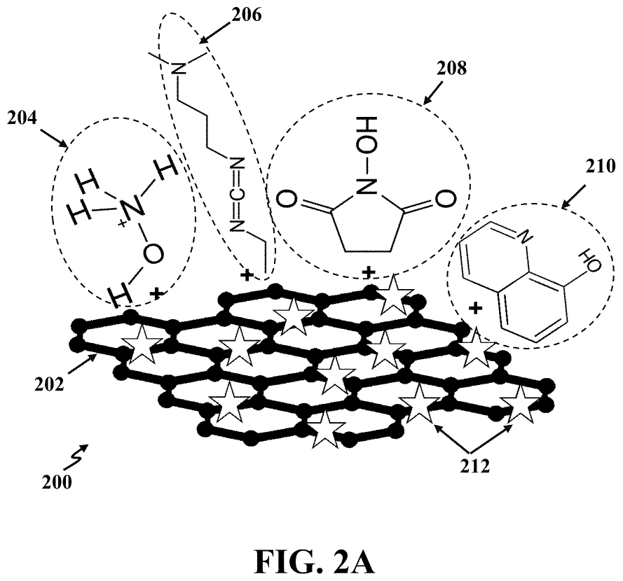

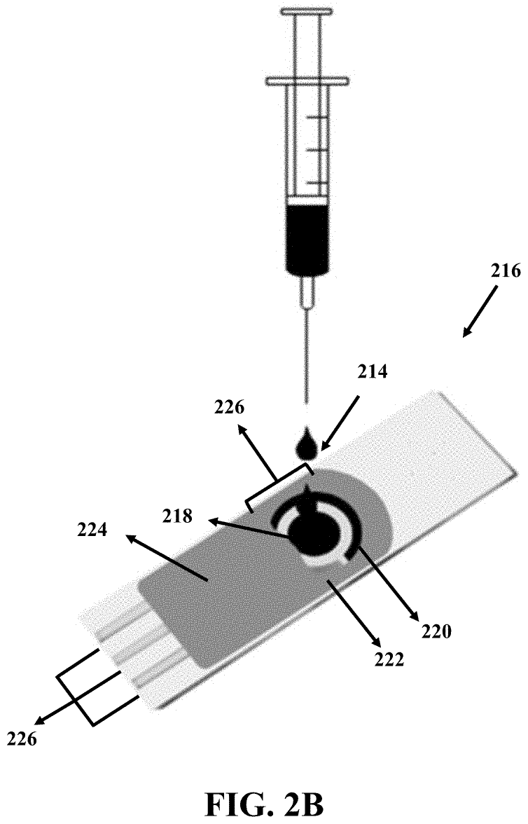

FIG. 2A illustrates a schematic of an exemplary label-free nanosensor 200 configured to detect glycoproteins in aqueous samples utilizing method 100 of FIG. 1, consistent with one or more exemplary embodiments of the present disclosure. In an exemplary embodiment, exemplary label-free nanosensor 200 may include a modified graphene oxide (GO) sheet and a signal amplifying agent 212 loaded onto the modified GO sheet. In an exemplary embodiment, the modified graphene oxide (GO) sheet may include a sensitive compound as a modifying agent conjugated to a GO sheet 202. In an exemplary embodiment, the modifying agent may include 1-ethyl-3-(3-dimethylaminopropyl) carbodiimide (EDC) 206, N-hydroxysuccinimide (NHS) 208, 8-hydroxyquinoline (8H) 210, and hydroxylammonium chloride 204. In an exemplary embodiment, exemplary label-free nanosensor 200 may include different functional groups on its surface, including an amine functional group, a carbonyl functional group, a hydroxyl functional group, and a methyl functional group. In an exemplary embodiment, different functional groups may be created on a surface of exemplary label-free nanosensor 200 due to conjugation of the modifying agent to GO sheet 202.

In an exemplary embodiment, the modifying agent may be conjugated to GO sheet 202 via at least one of a covalent bond or a hydrogen bond. In an exemplary embodiment, the modifying agent may be conjugated to GO sheet 202 via a covalent bond between functional groups of the GO sheets and functional groups of the modifying agent. In an exemplary embodiment, functional groups of the GO sheets may include hydroxyl groups and carboxyl groups.

In an exemplary embodiment, the modifying agent may include EDC 206 with a concentration between about 1% and about 20% by weight of the GO sheet, NHS 208 with a concentration between about 1% and about 20% by weight of the GO sheet, 8H 210 with a concentration between about 10% and about 50% by weight of the GO sheet, and hydroxylammonium chloride 204 with a concentration between about 10% and about 50% by weight of the GO sheet. In an exemplary embodiment, the modifying agent may further include cyclodextrin with a concentration between about 10% and about 50% by weight of the GO sheet. In an exemplary embodiment, the cyclodextrin may include at least one of α-cyclodextrin, β-cyclodextrin, and γ-cyclodextrin.

In an exemplary embodiment, signal amplifying agent 212 may be loaded onto the modified GO sheet via at least one of a covalent bond, a hydrogen bond, and an electrostatic interaction. In an exemplary embodiment, signal amplifying agent 212 may include at least one of an amine-functionalized gold nanoparticle and a silver nanoparticle. In the present disclosure, “amine-functionalized gold nanoparticle” refers to a gold nanoparticle functionalized with an amine group. In an exemplary embodiment, the amine-functionalized gold nanoparticle may include at least one of an amine-functionalized gold nanostar, an amine-functionalized gold nanorod, an amine-functionalized gold nanowire, an amine-functionalized gold spherical nanoparticle, an amine-functionalized gold nanoplate, and an amine-functionalized gold cubic nanostructure. In an exemplary embodiment, amine functional gold nanoparticles may have a size distribution between about 10 nm and about 100 nm.

In an exemplary embodiment, exemplary diagnostic kit may include screen-printed electrodes or fixed electrodes. FIG. 2B illustrates a schematic of putting an aqueous sample 214 in contact with exemplary diagnostic kit 216 (step 102), consistent with one or more exemplary embodiments of the present disclosure. Referring to FIG. 28, in an exemplary embodiment, diagnostic kit 216 may include screen-printed electrodes, including a working electrode 218, a counter electrode 220, a reference electrode 222. In an exemplary embodiment, working electrode 218 may include a plurality of label-free nanosensors 200 deposited (coated) on a substrate (not illustrated). In an exemplary embodiment, exemplary diagnostic kit 216 may further include an insulative layer 224 and a plurality of connectors 226. In an exemplary embodiment, aqueous sample 214 may be dropped on a sensing area 226 of exemplary diagnostic kit 216. In an exemplary embodiment, sensing area 226 may include working electrode 21, a counter electrode 220, a reference electrode 222.

In further detail with respect to step 104, in an exemplary embodiment, obtaining an electrochemical pattern may include recording the electrochemical pattern by applying an electrical potential to exemplary diagnostic kit. In an exemplary embodiment, applying an electrical potential to exemplary diagnostic kit may include applying a predetermined electrical potential between about −1 V and about 1 V to the diagnostic kit. In an exemplary embodiment, applying the electrical potential to the diagnostic kit may include applying the predetermined electrical potential between about −1 V and about 1 V with a scan rate between about 0.001 mV·s⁻¹ and about 0.05 mVs⁻¹ to the diagnostic kit.

In an exemplary embodiment, applying the electrical potential to exemplary diagnostic kit may include applying a predetermined electrical potential between about −0.5 V and about 0.5 V to the diagnostic kit. In an exemplary embodiment, obtaining the electrochemical pattern of the aqueous sample may include obtaining at least one of a cyclic voltammetry (CV) pattern, a differential pulse voltammetry (DPV) pattern, an electrochemical impedance spectroscopy (EIS) pattern, a square wave voltammetry (SWV) pattern, and a pattern of an amperometry assay of the aqueous sample. In an exemplary embodiment, the CV pattern may be obtained utilizing a cyclic voltammetry assay.

In an exemplary embodiment, upon applying the electrical potential to exemplary diagnostic kit, the aqueous sample's glycoproteins may be absorbed to exemplary label-free nanosensors of the working electrode. In an exemplary embodiment, applying an electrical potential to the diagnostic kit, may lead functional groups on hydrocarbon chains of glycoproteins to become capable of binding to functional groups of exemplary label-free nanosensor 200. In an exemplary embodiment, functional groups on hydrocarbon chains of glycoproteins may bind to functional groups of exemplary label-free nanosensor 200 through at least one of a covalent bond, a hydrogen bond, and an electrostatic interaction. In an exemplary embodiment, functional groups on hydrocarbon chains of glycoproteins may include at least one of hydroxyl groups, amine groups, methyl groups, and carbonyl groups.

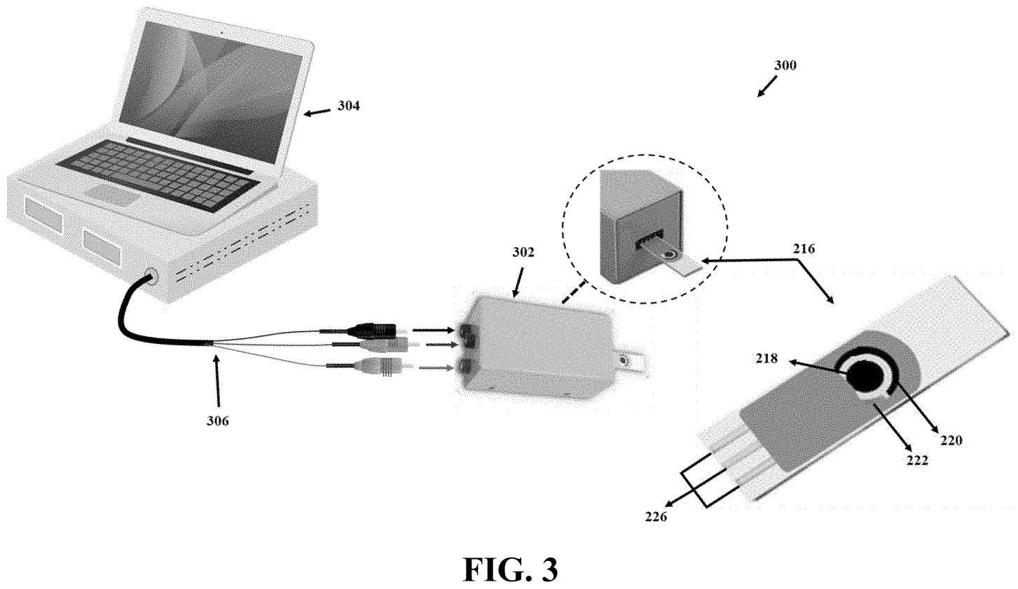

In an exemplary embodiment, applying the electrical potential to exemplary diagnostic kit may include applying a predetermined electrical potential to the diagnostic kit through an electrochemical system connected to the diagnostic kit. FIG. 3 illustrates a schematic of an exemplary electrochemical system 300 for detecting glycoproteins in aqueous samples, consistent with one or more exemplary embodiments of the present disclosure. In an exemplary implementation, system 300 may include a diagnostic kit 216, an electrochemical device 302, a processing unit 304, and a connection cable 306.

In an exemplary embodiment, diagnostic kit 216 may include three main electrodes, including a counter electrode 220, a reference electrode 222, and a working electrode coated with exemplary label-free nanosensor 218. Upon applying the potential to exemplary diagnostic kit 216 via electrochemical device 302 and connecting electrochemical device 302 to processing unit 304, system 300 may examine and identify glycoproteins' existence within an aqueous sample. Exemplary system 300 may also report each glycoprotein concentration based on a standard calibration curve of each glycoprotein.

In an exemplary embodiment, diagnostic kit 216 may be electrically connected to electrochemical device 302 via an electrical wire/cable or a wireless connection, and electrochemical device 302 may be electrically connected to processing unit 304 via electrical wires 306 or a wireless connection. In an exemplary embodiment, the wireless connection may include Bluetooth devices or Bluetooth modules embedded in diagnostic kit 216, electrochemical device 302, and processing unit 304. The wireless connection may allow for simplifying utilizing parts of system 300 at arbitrary distances from each other.

In an exemplary embodiment, electrochemical device 302 may include a potentiostat device. In an exemplary implementation, electrochemical device 302 may be configured to apply electrical potentials to exemplary diagnostic kit 216, measure electrical currents that may be generated between working electrodes 218 and counter electrode 220 respective to the applied electrical potentials, record the measured electrical currents respective to the applied electrical potentials, and send the recorded and measured electrical currents and applied electrical potentials to processing unit 304.

In an exemplary embodiment, processing unit 304 may be configured to record the electrochemical pattern based on the applied electrical potentials and the measured electrical current intensities, which may be sent by electrochemical device 302, calculate/measure the current intensity of the electrochemical pattern, and detect the glycoproteins in aqueous samples based on the electrochemical pattern in the aqueous sample. In an exemplary embodiment, processing unit 304 may further be configured to determine the type of the glycoprotein by looking up the voltage position of the electrochemical pattern of the aqueous sample in the database and measure the concentration of the glycoprotein in the database based on the calibration curve of the standard glycoprotein with the same voltage position.

In further detail with respect to step 106, in an exemplary embodiment, detecting a glycoprotein status of the aqueous sample may include detecting the glycoprotein status of the aqueous sample based on the electrochemical pattern of the aqueous sample. In an exemplary embodiment, detecting the glycoprotein status of the aqueous sample may include detecting that a glycoprotein may be present in the aqueous sample if the electrochemical pattern contains a peak and detecting that a glycoprotein may be absent in the aqueous sample if the electrochemical pattern lacks a peak.

In an exemplary embodiment, the peak may include a current intensity and a voltage position. In the present disclosure, a “peak” may refer to a point in an electrochemical pattern with a maximum current intensity in the Y-axis and a voltage position in the X-axis. The position of Y-axis is equal to the concentration of the glycoprotein and the position of X-axis is equal to the type of glycoprotein. In an exemplary embodiment, the maximum current intensity may include at least one of a local maximum intensity and a global maximum intensity. In an exemplary embodiment, the electrochemical pattern may have a domain which starts from one voltage position to another one and a peak is the climax at the highest height of the electrochemical pattern. Exemplary label-free nanosensor 200 may interact with active functional groups of glycoproteins in aqueous samples, leading to a differentiable electrochemical pattern at diverse voltage positions, which may be considered a fingerprint of each glycoprotein.

In an exemplary implementation, exemplary method 100 may further include identifying the glycoprotein in the aqueous sample by comparing a peak of an electrochemical pattern with standard peaks of standard electrochemical patterns in a database. FIG. 1B illustrates an exemplary implementation of exemplary method 100 for detecting glycoproteins in aqueous samples, consistent with one or more exemplary embodiments of the present disclosure. Referring to FIG. 1B, exemplary method 100 may include putting an aqueous sample in contact with exemplary diagnostic kit 216 (step 102), obtaining an electrochemical pattern by applying an electrical potential to exemplary diagnostic kit 216 (step 104), detecting a glycoprotein status of the aqueous sample based on the electrochemical pattern of the aqueous sample (step 106) and identifying the glycoprotein in the aqueous sample by comparing the peak of the electrochemical pattern with standard peaks of standard electrochemical patterns in a database (step 108).

In further detail with respect to step 108, in an exemplary embodiment, identifying die glycoprotein in the aqueous sample may include comparing the peak of the electrochemical pattern with standard peaks of standard electrochemical patterns in a database. In an exemplary embodiment, identifying the glycoprotein in the aqueous sample may include looking up the peak of the electrochemical patter of the aqueous sample in the database. In an exemplary embodiment, identifying the glycoprotein in the aqueous sample may include determining a type and a concentration of the glycoprotein in the aqueous sample.

In an exemplary embodiment, the database may include a plurality of datasets. In an exemplary embodiment, each dataset may be associated with a standard glycoprotein. In an exemplary embodiment, each dataset may include a standard electrochemical pattern of the standard glycoprotein and a calibration curve. In an exemplary embodiment, the standard electrochemical patter may include a standard peak, including a standard voltage position and a standard current intensity. In an exemplary embodiment, the calibration curve may relate the standard current intensity of the standard electrochemical pattern to a concentration of the standard glycoprotein.

FIG. 1C shows a flowchart of an exemplary method for comparing the peak of the electrochemical pattern with standard peaks of standard electrochemical patterns in the database, consistent with one or more exemplary embodiments of the present disclosure. Referring to FIG. IC, the exemplary process may be similar to step 108 of method 100, where the exemplary process may comprise of determining a type of the glycoprotein by finding a standard glycoprotein in the database through comparing the voltage position of the peak with standard voltage positions of the standard peaks in the database (step 110) and measuring a concentration of the glycoprotein based on the calibration curve of the standard glycoprotein (step 112).

In further detail with respect to step 110, in an exemplary embodiment, determining a type of the glycoprotein may include finding a standard glycoprotein in the database by comparing the voltage position of the peak with standard voltage positions of the standard peaks in the database. In an exemplary embodiment, finding the standard glycoprotein in the database may include looking up a standard glycoprotein similar to the glycoprotein regarding the peak's voltage position in the database.

In an exemplary embodiment, upon applying the potential to the aqueous sample, exemplary label-free nanosensor 200 deposited on the substrate of working electrode 218 may absorb the glycoproteins to itself via functional groups on the surface of exemplary label-free nanosensor. In an exemplary embodiment, working electrode 218 may generate a unique electrochemical pattern for each examined glycoprotein through an electrochemical assay. In an exemplary embodiment, functional groups of exemplary label-free nanosensor may include at least one of a carbonyl group, a hydroxyl group, a methyl group, and an amine group. In an exemplary embodiment, interactions between glycoproteins and exemplary label-free nanosensor may be performed via confined-surface reactions and adsorption electron transfer process on the surface of working electrode 218. In an exemplary embodiment, the reaction between glycoproteins and exemplary label-free nanosensor may be performed via an electrochemical (E) mechanism.

In further detail with respect to step 112, in an exemplary embodiment, measuring the glycoprotein concentration may include measuring the glycoprotein concentration based on the calibration curve of the standard glycoprotein. In an exemplary embodiment, measuring the glycoprotein concentration based on the calibration curve of the standard glycoprotein may include measuring the concentration of the glycoprotein based on the calibration curve of the standard glycoprotein similar to the glycoprotein regarding the voltage position of the peak.

In an exemplary embodiment, the calibration curve may relate the standard current intensity of the standard electrochemical pattern to a concentration of the standard glycoprotein. The calibration curve may relate the standard current intensity of the standard electrochemical pattern to a concentration of the standard glycoprotein. In an exemplary embodiment, the current intensity may be directly proportional to the concentration of the glycoprotein. In an exemplary embodiment, the current intensity may be increased concerning an incase in glycoproteins' concentration.

In an exemplary embodiment, a calibration curve may be obtained upon diluting a standard stock of a glycoprotein's sample and obtaining the electrochemical intensity of different concentrations of the target glycoprotein structure within the PBS. In an exemplary embodiment, the calibration curve may generate a linear relationship between the target glycoprotein concentration and the intensity obtained from the electrochemical system. In an exemplary embodiment, the glycoprotein concentration may be calculated by finding a concentration related to an intensity obtained from the electrochemical pattern of the target glycoprotein in the aqueous sample in the standard calibration curve of that particular glycoprotein.

In an exemplary implementation, exemplary system 300 may be utilized for carrying out obtaining an electrochemical pattern by applying an electrical potential to exemplary diagnostic kit 216 (step 104) and detecting a glycoprotein status of the aqueous sample based on the electrochemical pattern of the aqueous sample (step 106), and identifying the glycoprotein in the aqueous sample by comparing the peak of the electrochemical pattern with standard peaks of standard electrochemical patterns in a database (step 108).

In an exemplary implementation, exemplary method 100 may further include generating a database including a plurality of datasets of a plurality of standard glycoprotins. FIG. 1D illustrates another exemplary implementation of exemplary method 100 of FIG. 1B for detecting glycoproteins in aqueous samples, consistent with one or more exemplary embodiments of the present disclosure. Referring to FIG. 1D, exemplary method 100 may include generating a database including a plurality of datasets of a plurality of standard glycoproteins (step 101), putting an aqueous sample in contact with exemplary diagnostic kit 216 (step 102), obtaining an electrochemical pattern by applying an electrical potential to exemplary diagnostic kit 216 (step 104), detecting a glycoprotein status of the aqueous sample based on the electrochemical pattern of the aqueous sample (step 106), and identifying the glycoprotein in the aqueous sample by comparing the peak of the electrochemical pattern with standard peaks of standard electrochemical patterns in a database (step 108).

In further detail with respect to step 101, in an exemplary embodiment, generating a database may include generating the database including a plurality of datasets of a plurality of standard glycoproteins. In an exemplary embodiment, the database may include a plurality of datasets. In an exemplary embodiment, each dataset may be associated with a standard glycoprotein. In an exemplary embodiment, each dataset may include a standard electrochemical pattern of the standard glycoprotein and a calibration curve. In an exemplary embodiment, the standard electrochemical pattern may include a standard peak, including a standard voltage position and a standard current intensity. In an exemplary embodiment, the calibration curve may relate the standard current intensity of the standard electrochemical pattern to a concentration of the standard glycoprotein.

FIG. 1E illustrates an exemplary method for generating a database including a plurality of datasets of a plurality of standard glycoproteins, consistent with one or more exemplary embodiments of the present disclosure. Referring to FIG. 1E, the exemplary process may be similar to step 101 of method 100, where the exemplary process may comprise of obtaining a plurality of standard electrochemical patterns of a plurality of standard glycoproteins (step 114) and plotting a calibration curve for each standard glycoprotein pattern by relating the standard current intensity of each standard electrochemical pattern to a concentration of the standard glycoprotein (step 116).

In further detail with respect to step 114, in an exemplary embodiment, obtaining a plurality of standard electrochemical patterns of a plurality of standard glycoproteins may include putting a plurality of standard solutions of a standard glycoprotein in contact with exemplary diagnostic kit 216 and obtaining a standard electrochemical patterns of the standard glycoproteins by applying an electrical potential to exemplary diagnostic kit. In an exemplary embodiment, the plurality of standard solutions of a standard glycoprotein may include standard solutions with different concentrations of the standard glycoprotein. In the present disclosure, “a standard glycoprotein” may include a glycoprotein whose unique electrochemical pattern and its calibration curve are obtained and entered into the database. In the present disclosure. “standard solution of a standard glycoprotein” refers to a solution that includes an electrochemical pattern with a peak specific to the standard glycoprotein. In an exemplary embodiment, a standard solution of a standard glycoprotein may be obtained by adding the standard glycoprotein to a solution with no electrochemical peak. In an exemplary embodiment, each standard electrochemical pattern of the standard glycoprotein may include a standard peak, including a standard voltage position and a standard current intensity. In an exemplary embodiment, each standard glycoprotein may have a unique electrochemical pattern.

In further detail with respect to step 116, in an exemplary embodiment, plotting a calibration curve for each standard glycoprotein pattern may include relating the standard current intensity of each standard electrochemical pattern to a concentration of the standard glycoprotein. In an exemplary embodiment, plotting the calibration curve for each standard glycoprotein pattern may include plotting the calibration curve for standard solutions of each standard glycoprotein pattern by relating the standard current intensity of each standard electrochemical pattern to a concentration of each standard solution of the standard glycoprotein.

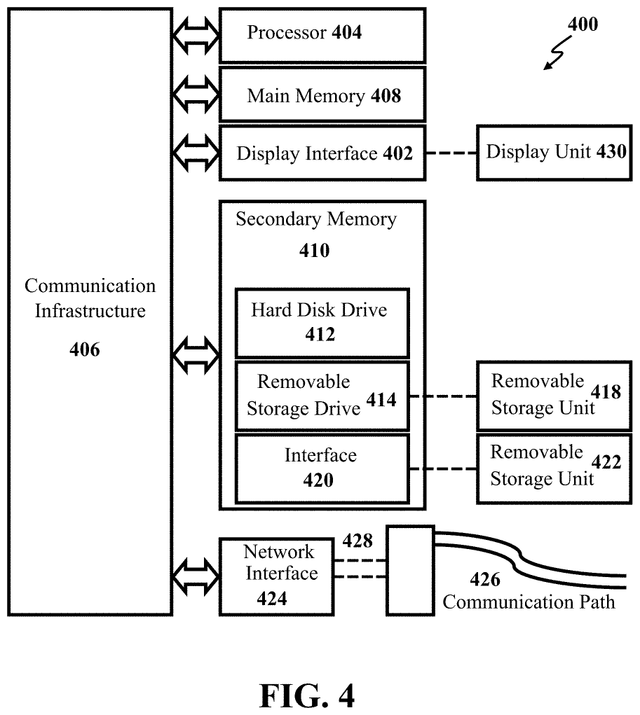

FIG. 4 illustrates an exemplary computer system 400 in which an embodiment of the present disclosure, or portions thereof, may be implemented as computer-readable code, consistent with one or more exemplary embodiments of the present disclosure. For example, steps 101, 104, 106, and 108 of flowcharts presented in method 100 may be implemented in computer unit 400 using hardware, software, firmware, tangible computer-readable media having instructions stored thereon, or a combination thereof and may be implemented in one or more. Hardware, software, or any combination may embody any of the modules and components in FIGS. 1A-3.

If programmable logic is used, such logic may execute on a commercially available processing platform or a particular purpose device. One ordinary skill in the art may appreciate that an embodiment of the disclosed subject matter can be practiced with various processor configurations, including multi-core multiprocessor systems, minicomputers, mainframe computers, computers linked or clustered with distributed functions, as well as pervasive or miniature computers that may be embedded into virtually any device.

For instance, a computing device with at least one processor device and a memory may implement the above-described embodiments. A processor device may be a single processor, a plurality of processors, or combinations thereof. Processor devices may have one or more processor “cores.”

An embodiment of the invention is described in terms of this example computer unit 400. After reading this description, it may become apparent to a person skilled in the relevant art how to implement the invention using other processors and/or computer architectures. Although operations may be described as a sequential process, some of the operations may be performed in parallel, concurrently, and/or in a distributed environment, and with program code stored locally or remotely for access by single or multiprocessor machines. In addition, in some embodiments, the order of operations may be rearranged without departing from the spirit of the disclosed subject matter.

Processor device 404 may be a special purpose or a general-purpose processor device. As may be appreciated by persons skilled in the relevant art, processor device 404 may also be a single processor in a multi-core/multiprocessor system, such system operating alone or in a cluster of computing devices operating in a cluster or server farm. Processor device 404 may be connected to a communication infrastructure 406, for example, a bus, message queue, network, or multi-core message-passing scheme.

In an exemplary embodiment, computer unit 400 may include a display interface 402, for example, a video connector, to transfer data to a display unit 430, for example, a monitor. Computer unit 400 may also include a main memory 408, for example, random access memory (RAM), and may also include a secondary memory 410. Secondary memory 410 may include, for example, a hard disk drive 412 and a removable storage drive 414. Removable storage drive 414 may include a floppy disk drive, a magnetic tape drive, an optical disk drive, a flash memory, or the like. Removable storage drive 414 may read from and/or write to a removable storage unit 418 in a well-known manner. Removable storage unit 418 may include a floppy disk, a magnetic tape, an optical disk, etc., which may be read by and written to by removable storage drive 414. As will be appreciated by persons skilled in the relevant art, removable storage unit 418 may include a computer-usable storage medium having stored therein computer software and/or data.

In alternative implementations, secondary memory 410 may include other similar means for allowing computer programs or other instructions to be loaded into computer unit 400. Such means may include, for example, a removable storage unit 422 and an interface 420. Examples of such means may include a program cartridge and cartridge interface (such as that found in video game devices), a removable memory chip (such as an EPROM, or PROM) and associated socket, and other removable storage units 422 and interfaces 420, which allow software and data to be transferred from removable storage unit 422 to computer unit 400.

Computer unit 400 may also include a communications interface 424. Communications interface 424 allows software and data to be transferred between computer unit 400 and external devices. Communications interface 424 may include a modem, a network interface (such as an Ethernet card), a communications port, a PCMCIA slot, card, or the like. Software and data transferred via communications interface 424 may be in the form of signals, which may be electronic, electromagnetic, optical, or other signals capable of being received by communications interface 424. These signals may be provided to communications interface 424 via a communications path 426. Communications path 426 carries signals and may be implemented using wire or cable, fiber optics, a phone line, a cellular phone link, an RF link, or other communications channels.

In this document, the terms “computer program medium” and “computer usable medium” are used to generally refer to media such as removable storage unit 418, removable storage unit 422, and a hard disk installed in hard disk drive 412. Computer program Tedium and computer usable medium may also refer to memories, such as main memory 408 and secondary memory 410, which may be memory semiconductors (e.g. DRAMs, etc.).

Computer programs (also called computer control logic) are stored in main memory 408 and/or secondary memory 410. Computer programs may also be received via communications interface 424. Such computer programs, when executed, enable computer unit 400 to implement different embodiments of the present disclosure as discussed herein. In particular, the computer programs, when executed, enable processor device 404 to implement the processes of the present disclosure, such as the operations in method 100 illustrated by flowchart 100 of FIG. 1A discussed above. Accordingly, such computer programs represent controllers of computer unit 400. Where an exemplary embodiment of method 100 is implemented using software, the software may be stored in a computer program product and loaded into computer unit 400 using removable storage drive 414, interface 420, and hard disk drive 412, or communications interface 424.

Embodiments of the present disclosure also may be directed to computer program products, including software stored on any computer useable medium. Such software, when executed in one or more data processing devices, causes a data processing device to operate as described herein. An embodiment of the present disclosure may employ any computer useable or readable medium. Examples of computer useable mediums include, but are not limited to, primary storage devices (e.g., any type of random-access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

### EXAMPLES

### Example 1: Fabrication of Exemplary Label-Free Nanosensor for Glycoprotein Detection in

In this example, exemplary label-free nanosensor, as illustrated in FIG. 2, was fabricated using a fabrication method. The fabrication method included producing modified graphene oxide (GO) sheets and preparing a working electrode by depositing a mixture of modified GO sheets and amine-functionalized gold nanostars (Au NSs) on a screen-printed carbon electrode (SPCE). The modified GO sheets were produced by conjugating EDC, NHS, 8H, and hydroxylammonium chloride as modifying agents to GO sheets.

At first, a homogenous suspension of GO sheets was obtained by adding well-exfoliated GO sheets with an amount of about 50 g to tetrahydrofuran (THF) with a volume of about 5 L, ultrasonication at 600 W for a time period of about 30 minutes followed by mixing at a speed of about 2000 rpm for 24 hours. The resulting homogenous suspension of GO sheets was poured into a 50 L vessel equipped with a heating belt. After that, a first mixture was obtained by evaporating the THF from the homogenous suspension of GO sheets by adding ultrapure degassed water with a volume of about 25 L to the homogenous suspension of GO and ultrasonication at 600 W for a time period between about 10 minutes and about 60 minutes at a temperature between about 80° C. and about 100° C.

In the next step, a second mixture was obtained by modifying the GO sheets through mixing EDC with a concentration of about 5 wt. % of the weight of the GO sheet, NHS with a concentration of about 5 wt. % of the weight of the GO sheet, 8H with a concentration of about 20 wt. % of the weight of the GO sheet with the suspension of GO sheets for a time period of about 1 hour at a speed of about 1000 rpm under reflux. The hydroxyl ammonium chloride with a concentration of about 20 wt. % of the weight of the GO sheet was also mixed with the second mixture for a time period of about 1 hour.

In the next step, modified GO sheets were obtained by dropwise adding ammonia with a volume between about 1 L and 2 L to the second mixture and mixed for 24 hours under reflux. In the end, the modified GO sheets were filtrated using a polytetrafluoroethylene (PTFE) filter bag with a pore size of about 0.22 μm under reducing pressure generated utilizing a vacuum pump. The modified GO sheets were also well-washed with deionized water and dried in an oven at a temperature between about 60° C. and 80° C. for a time period of about 12 hours and stored in a desiccator to be further used.

In the next step, the working electrode was prepared by depositing exemplary label-free nanosensor, including a mixture of modified GO sheets and amine-functionalized Au NSs on the substrate. In one or more exemplary embodiments, depositing exemplary label-free nanosensor including a mixture of modified GA sheets and Au NSs on the substrate may be accomplished using deposition methods, including at least one of drop-casting, dip-coating, spin coating, blade coating, electrochemical deposition, electrospinning deposition, electrospray deposition, physical vapor deposition, chemical vapor deposition, screen printing, inkjet printing, nozzle-jet printing, and laser scribing.

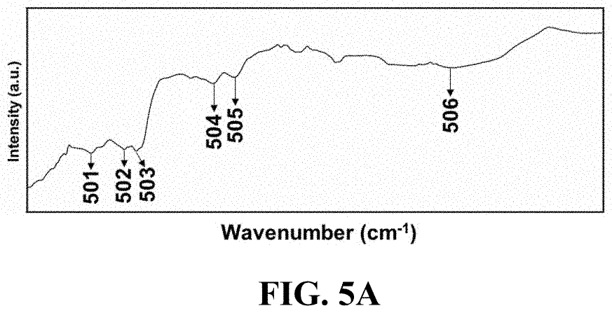

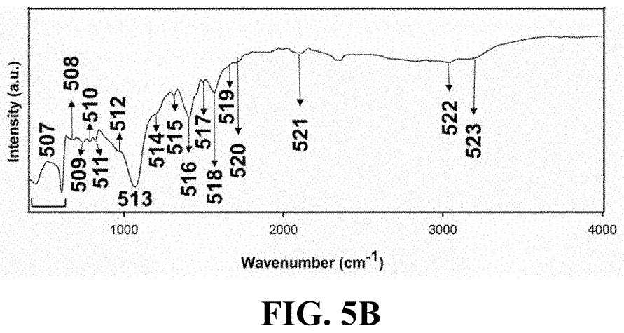

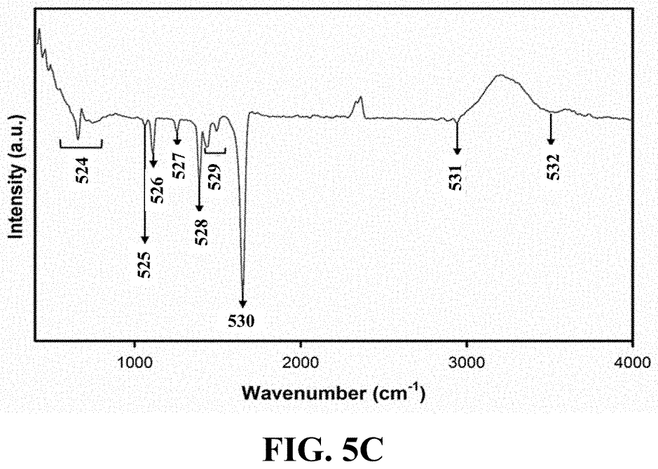

FIG. 5A illustrates a Fourier-transform infrared (FTIR) spectrum of graphene oxide (GO) sheets, consistent with one or more embodiments of the present invention. Referring to FIG. 5A, GO sheets included common functional groups, including sp²=C—H 500, in-plane C—H vibration 502, CO alkoxy 504, C═C double bond carbon atoms 506, C═O carbonyl functional group (sp³ hybridization) 505, and hydroxyl functional groups (—OH) 508. FIG. 5B illustrates an FTIR spectrum of modified GO sheets, including GO sheets modified with 1-ethyl-3-(3-dimethylaminopropyl) carbodiimide (EDC), N-hydroxysuccinimide (NHS), 8-hydroxyquinoline (8H), and hydroxylammonium chloride, consistent with one or more embodiments of the present invention.

Referring to FIG. 5B, GO sheets were successfully modified with common functional groups including sp² C—H 501, in-plane C—H vibration 502, CO alkoxy 503, C═C double bond carbon atoms 504. C═O carbonyl functional group (sp³ hybridization) 505, and hydroxyl functional groups (—OH) 506. Referring to FIG. 5B, each appeared peak is attributed to the deformation of 8H's benzene ring 507, torsion of benzene ring of 8H 508, —OCN out of plane asymmetric defect related to NIS 509, C—H bending of 8H 510, out of plane bending of ═CH₂ and ═C—H functional groups of 8H on the surface of GO 511, out of plane deformation of CH₃ functional group of 8H 512, 1 substitution of aromatic benzene ring related to 8H 513. C—H stretching vibration of 8H 514, asymmetric stretching vibration of —CNC— attributed to presence of NHS 515, ring stretching vibration due to the vibration of O—C functional group of H 516, α-CH₃ bending arised from 8H 517, in plane deformation of CH₂ functional group of 8H 518, C═C double bond carbon atoms which known as the finger print of GO 519. CO stretch of amide related to NHS 520, asymmetric stretching vibration of —C═O due to the presence of NHS on the surface of GO 521, highly active —N═C═N— functional groups of EDC 522, hydroxyl functional group and integrated NH stretching vibration with hydrogen bonding of NHS along —NH bridge of either 8H and hydroxyl ammonium chloride on the surface of GO 523.

Also, amine-functionalized gold nanostars (Au NSs) were synthesized using a chloroauric acid (HAuCl₄) suspension. First, a primary stock was prepared by dissolving a 0.25 M suspension of HAuCl₄ in about 420 mL dimethylformamide (DMF). In the next step, about 20-30 mL of polyvinylpyrrolidone (PVP) suspension (1-10 wt % dissolved within DMF), about 1-10 mL diethylamine, and 1-10 mL of the primary stock of the HAuCl₄ suspension were added to 420 mL DMF and mixed (1000 rpm) for 5-10 minutes at room temperature; as a result, the color of the suspension was changed from deep yellow to clear. Then, Au NSs were synthesized by mixing the resulting suspension (500-1000 rpm) at 110° C. for about 10-20 minutes; thus, the suspension color was changed from clear to brownish blue. The resulting Au NSs were centrifuged at 5000-10000 rpm for 30-60 minutes. Then, the supernatant was removed, and deionized water was added to sedimented Au NSs. The resulting suspension containing Au NSs was ultrasonicated at 400-600 W for 10-30 minutes and stored at 4° C. for further use.

FIG. 5C illustrates an FTIR spectrum of gold nanostars (Au NS), consistent with one or more embodiments of the present disclosure. FIG. 5C shows the FTIR spectrum of Au NSs, consistent with one or more embodiments of the present disclosure. Referring to FIG. 5C, appeared peaks corresponding to N—H may be considered as primary and secondary amines 524, C-A stretching vibration (alkyl ether) 525, C—N stretching vibration of diethylamine 526, C—N stretching vibration of aliphatic amine functional groups 527, O—H bending of phenol groups 52g, C—H stretching vibration 529, N—H bending of primary amine of diethylamine 530, C—H stretching vibration of alkane groups 531 and O—H stretching vibration as a result of Au reduction to Au⁰ 532. These appeared peaks confirm the successful fabrication of the Au NSs.

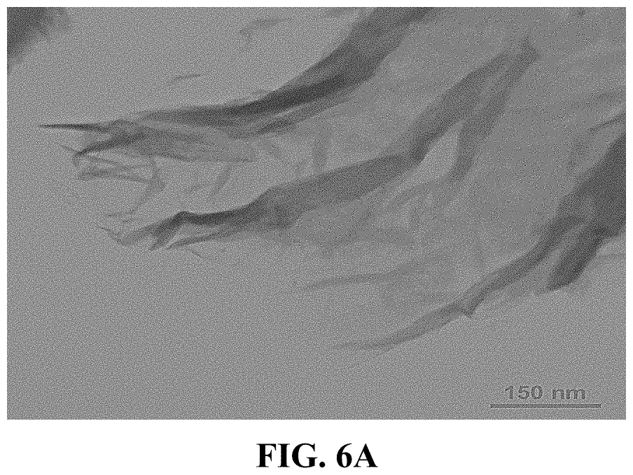

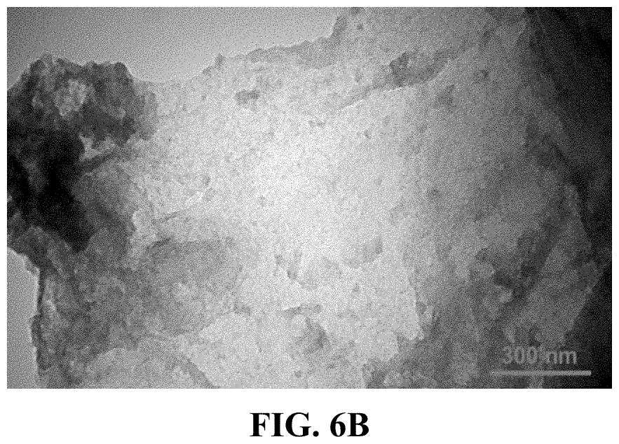

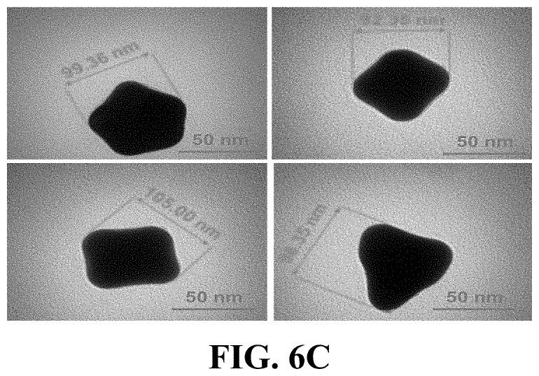

FIG. 6A illustrates a transmission electron microscopy (TEM) image of GO sheets, consistent with one or more embodiments of the present disclosure. FIG. 68 illustrates a TEM image of modified GO sheets, including GO sheets modified with EDC, NHS, 8H, and hydroxylammonium chloride, consistent with one or more embodiments of the present disclosure. Referring to FIGS. 6A-6B, it may be concluded that the graphene oxide was correctly generated from well-exfoliated graphite sheets and presenting a wide active surface area for homogeneous distribution of active chemical compounds. Correspondingly. GO sheets modified with EDC. NHS, 8H, and hydroxylammonium chloride also exhibiting a well-resolved 2D nanosheet with homogenously dispersed modifying agents throughout the active surface area of the GO sheets. FIG. 6C illustrates TEM images of the gold nanostars (Au NS), consistent with one or more embodiments of the present disclosure. Referring to FIG. 6C. Au NSs were successfully synthesized with well-defined star-shaped morphology and uniform size distribution. This star-shaped morphology and active functional groups of Au NSs provide the possibility for either covalent or hydrogen binding with the modified GO sheet and thus improved interaction for the final integrated platform with glycoprotein structures.

In the end, a working electrode was produced as follows. At first, a mixture of modified GO sheets was prepared by dissolving the modified GO sheets in ultra-pure deionized water with a concentration of about 2 mg/ml. An active suspension was then formed by mixing the mixture of modified GO sheets with an equal volume ratio of Au NS suspension. In the end, a working electrode of each SPCE was coated with 5 μL of the active suspension and scaled with sulfonated tetrafluoroethylene-based fluoropolymer-copolymer to avoid detachment of exemplary label-free nanosensor from the working electrode.

### Example 2: Electrochemical Evaluation of Exemplary Label-Free Nanosensor

In this example, electrochemical features, including CV and EIS patterns, of a diagnostic kit, similar to exemplary diagnostic kit 216, were evaluated utilizing an electrochemical system, similar to exemplary electrochemical system 300. The diagnostic kit included a working electrode, the working electrode included label-free nanosensors, similar to exemplary label-free nanosensor 200. The CV and EIS patterns were recorded using S mM (Fe(CN)₆)³⁻⁴ solution containing 0.1 M KCl at a scan rate of 100 mV s⁻¹.

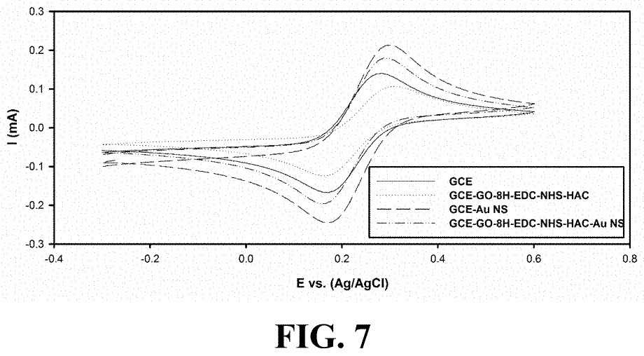

FIG. 7 illustrates a result of cyclic voltammetry (CV) analysis of glassy carbon electrode (GCE) as an unmodified working electrode, and GCE deposited with modified GO sheets (GCE-GO-8H-EDC-NHS-HAC), GCE deposited with Au NSs (GCE-AuNS), and exemplary label-free nanosensor (GCE-GO-8H-EDC-NHS-HAC-AuNS), consistent with one or more embodiments of the present disclosure. Referring to FIG. 7, when the modified GO sheets (GO-8H-EDC-NHS-HAC) as the label-free nanosensors were deposited (coated) on the GCE, the height of peak currents decreased. Also, the peak separation occurred due to the formation of insulating layers of GO-8H-EDC-NHS-HAC, which increased the rate of insulative hydroxyl functional groups on the electrode's surface. However, peak currents (Ip) of (Fe(CN)₆)³⁻⁴ redox probe were remarkably enhanced upon introduction of Au NS that may be due to the excellent conductivity and ability of Au NSs to facilitate the charge transfer within the solution.

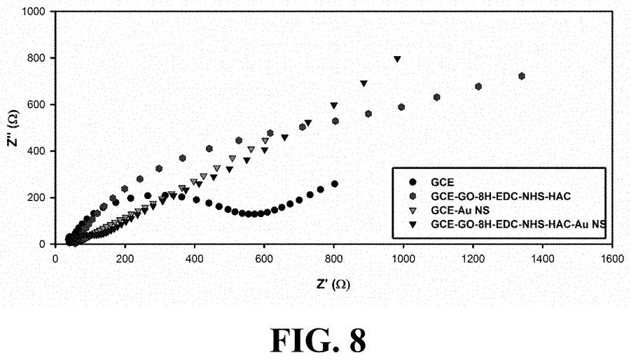

FIG. 8 illustrates a result of electrochemical impedance spectroscopy (EIS) analysis of glassy carbon electrode (GCE) as an unmodified working electrode, and GCE deposited with modified GO sheets (GCE-GO-8H-EDC-NHS-HAC), GCE deposited with Au NSs (GCE-AuNS), and exemplary label-free nanosensor (GCE-GO-8H-EDC-NHS-HAC-AuNS), consistent with one or more embodiments of the present disclosure. Referring to FIG. 8, the unmodified working electrode (GCE) showed a charge-transfer resistance (Rₑₜ) value of 554Ω, whereas the GCE deposited with Au NS (GCE-AuNS) showed a straight line with a low R value of 21.53Ω a representing a more straightforward charge transfer process due to the predominant ability of Au NS for electronic transfer. The GCE deposited with modified GO sheets (GO-8H-EDC-NHS-HAC) exhibited higher resistance (Rₑₜ of 1130Ω); whereas, modifying the working electrode with exemplary label-free nanosensor (GO-8H-EDC-NHS-HAC-AuNS) resulted in a relatively low Rₑₜ with a small semicircle domain (R_{D}=83.64Ω). This drastic change is due to the electrocatalytic properties of Au NSs as a superiorly conductive and electroactive nanomaterial. The obtained results may indicate that modification of the GCE with exemplary label-free nanosensor (GO-8H-EDC-NIS-HAC-AuNS) may considerably increase the electron communication features of the unmodified working (GCE).

### Example 3: Detection of Infectious Bronchitis Virus (IBV) Using Exemplary Diagnostic Kit

In this example, detection of glycoproteins of infectious bronchitis virus (IBV) in different samples was done using exemplary diagnostic kit, similar to exemplary diagnostic kit 216. Evaluation of the exemplary diagnostic kit performance was done by dropping about 100 μL of a biological sample, for instance, saliva, containing IBV onto the top surface of the working electrode, the counter electrode, and the reference electrode. A voltage ranging from −0.2 to 0.2 V, particularly −0.1 to 0.1 V, was applied to the diagnostic kit, and the DPV pattern of each sample was monitored following 30 to 60 seconds.

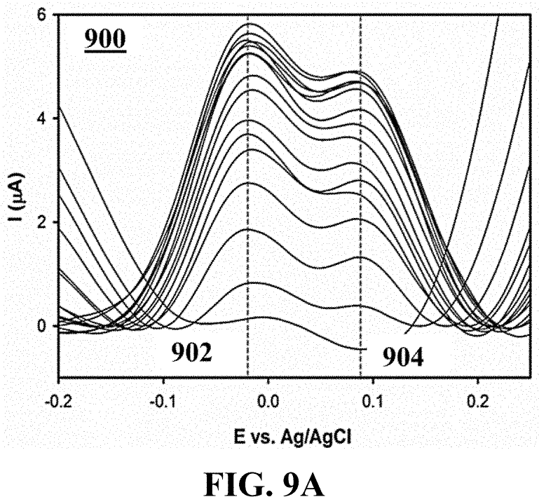

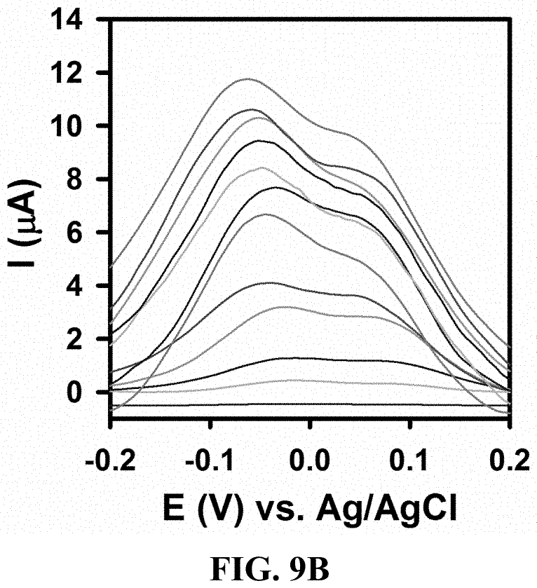

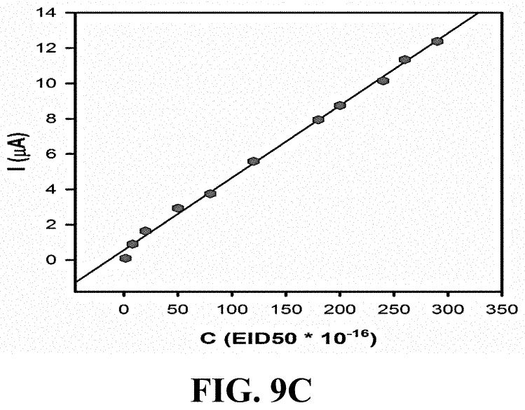

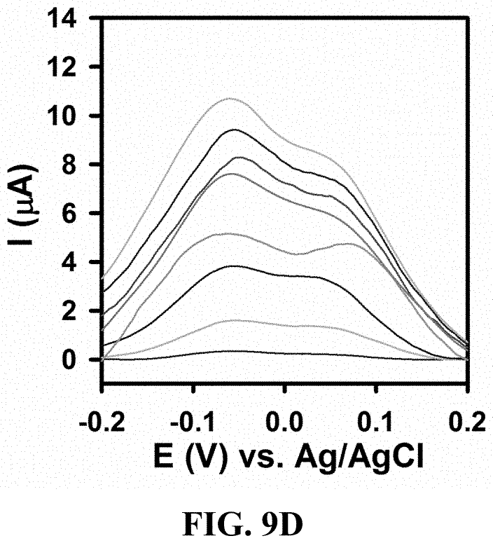

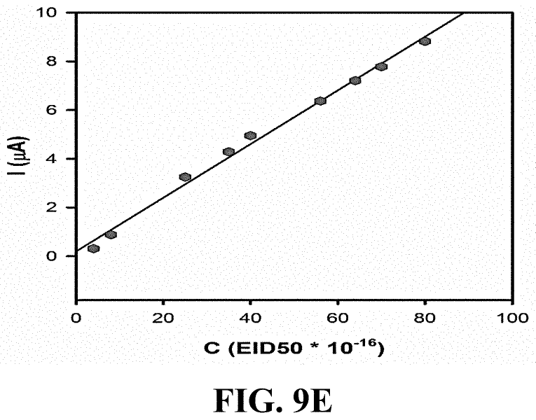

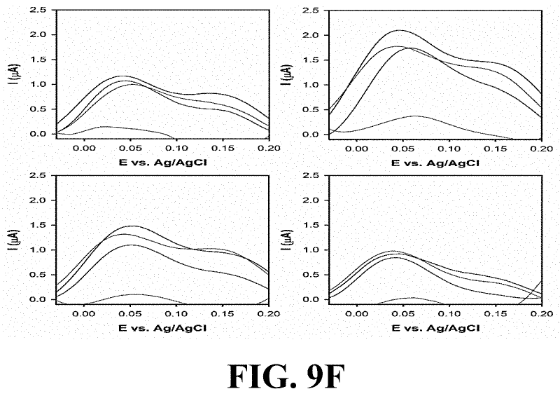

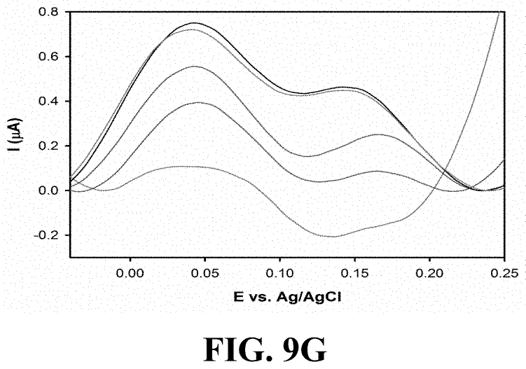

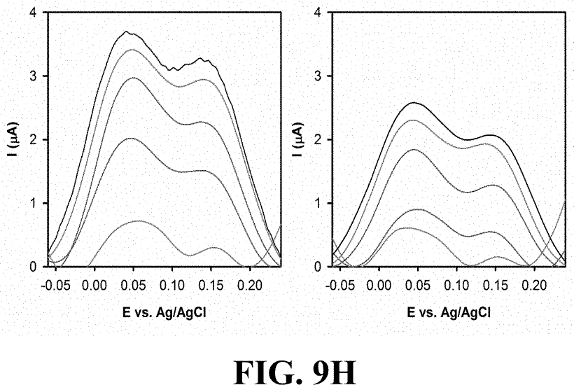

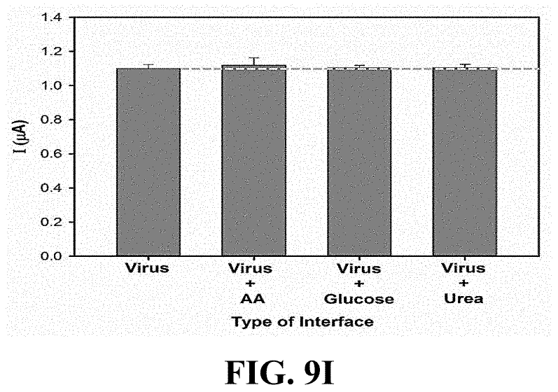

FIG. 9A illustrates a differential pulse voltammetry (DPV) pattern of whole glycoproteins of infectious bronchitis virus (IBV) 900, consistent with one or more embodiments of the present disclosure. Referring to FIG. 9A, due to the higher amount of S spike glycoprotein on the surface of IBV, both peaks 902 and 904 may be generated owing to the interaction of exemplary label-free nanosensor with S spike glycoproteins of IBV. FIG. 9B illustrates a DPV pattern of spike glycoprotein of IBV in phosphate-buffered solution (PBS), consistent with one or more embodiments of the present disclosure. Referring to FIG. 9B, an increase in the concentration of viruses in the aqueous sample may increase S spike glycoprotein's corresponding peak, which may be used as a metric to draw the related calibration curve and measure the exact population of viruses in aqueous matters. FIG. 9C illustrates a calibration curve of IBV in PBS, consistent with one or more embodiments of the present disclosure. Referring to FIG. 9C, an exact concentration of IBV may be detected upon changing the intensity of detected peaks based on 1 (μA).

FIG. 9D illustrates a DPV pattern of spike glycoprotein of IBV in a human blood plasma sample, consistent with one or more embodiments of the present disclosure. FIG. 9E illustrates a calibration curve of IBV in a human blood plasma sample, consistent with one or more embodiments of the present disclosure. Referring to FIGS. 9D-9E, it may be concluded that exemplary label-free nanosensor detected the related glycoprotein structure of IBV in the same voltage position, which furtherly highlighting the efficient performance of exemplary label-free nanosensor toward detection of pathogenic viruses like IBV.

Also, an exact concentration of IBV may be detected upon a change in the intensity of detected peaks based on I (μA).

FIG. 9F illustrates DPV patterns of IBV in oropharyngeal swabs of chickens infected with wild type IBV, consistent with one or more embodiments of the present disclosure. Referring to FIG. 9F, all extracted oropharyngeal swabs showed the unique electrochemical pattern of IBV corresponding to S glycoprotein of coronavirus at around 0.04 and 0.14 V, respectively.

Performance of exemplary diagnostic kit was also evaluated by detecting IBV glycoproteins in the tracheal mucosa layer extracted from an infected bird with a wild-type strain of IBV. To this end, the swab was placed within 1 mL PBS (pH=7.4) and kept stationary till the extraction of viruses from the tissue. Afterward, the sample was shaken for 10 min, and a trace of the virus was detected within the aquatic media. FIG. 9G illustrates a DPV pattern of 1V in a tracheal mucosa layer extracted from an infected bird with a wild-type strain of IBV, consistent with one or more embodiments of the present disclosure. Referring to FIG. 9G, IBV was successfully extracted from the tissue into the aquatic media, and a unique electrochemical pattern of the IBV was detected via exemplary diagnostic kit. The unique electrochemical pattern of IBV was in accord with previous analyses, and their peaks appeared at voltage positions of about 0.04 V and about 0.14 V. FIG. 9H illustrates DPV patters of IBV in extracted blood samples from infected chickens with the wild-type strain of IBV, consistent with one or more embodiments of the present disclosure. Referring to FIG. 9H, both blood samples showed a trace of IBV glycoproteins at the same peaks at the voltage position of about 0.04 V and about 0.14 V, which corresponded to the unique electrochemical viral pattern glycoproteins of IBV and confirmed the existence of IBV in the samples.

Moreover, the overall influence of typical electroactive interferences within the real biological fluid on current responses of 2.0×10¹⁴ median embryo infectious dose (EID50) coronavirus was investigated via adding 0.1 mM interfering biomolecules such as ascorbic acid (AA), glucose, and urea. FIG. 9I illustrates the effect of electroactive interfaces on the DPV pattern obtained using exemplary diagnostic kit, consistent with one or more embodiments of the present disclosure. Referring to FIG. 9I, no change was observed in the Iₚ of viral spike (S) glycoproteins demonstrating the predominant ability of exemplary diagnostic kit for precise detection of viral glycoproteins of IBV in the presence of possible interfering compounds.

### Example 4: Detection of SARS-CoV-2 Using Exemplary Diagnostic Kit

In this example, detection of glycoproteins of severe acute respiratory syndrome coronavirus-2 (SARS-CoV-2) in different samples was done using exemplary diagnostic kit, similar to exemplary diagnostic kit 216. Also, an electrochemical system similar to electrochemical system 300, including exemplary diagnostic kit 216, was utilized to process an exemplary method similar to method 100 for testing the presence of SARS-CoV-2 glycoproteins in normal (not-infected with SARS-CoV-2) and infected cases with SARS-CoV-2.

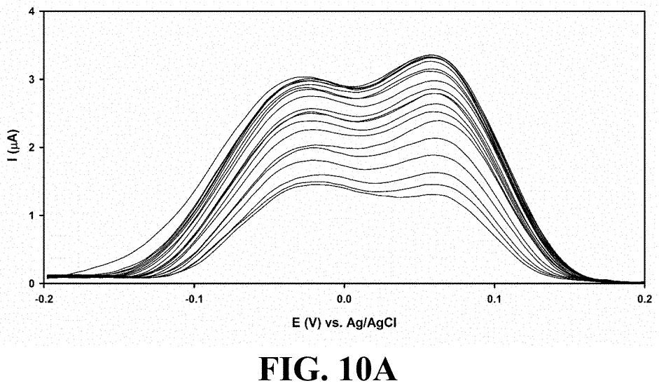

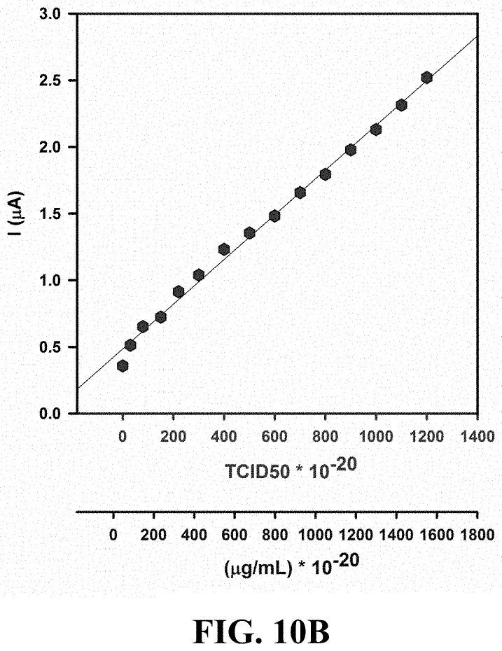

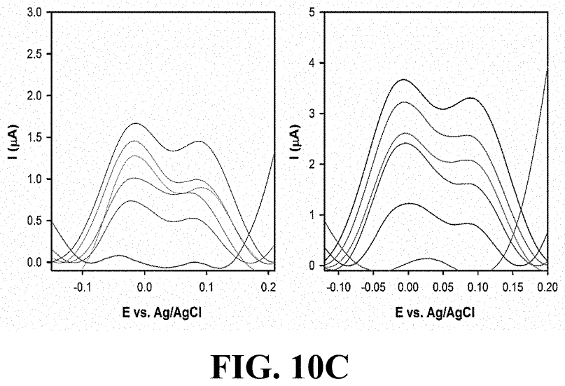

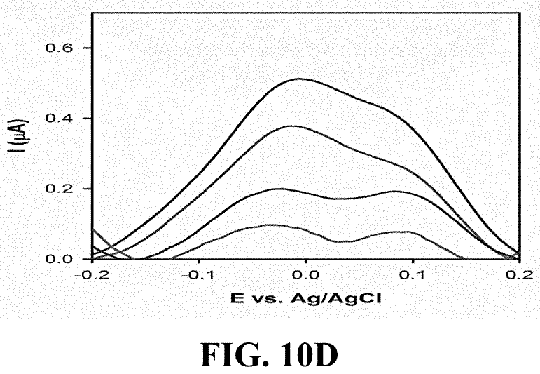

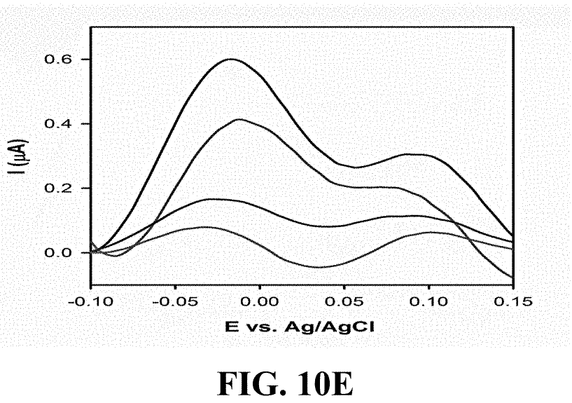

FIG. 10A illustrates the DPV pattern of SARS-CoV-2 in PBS, consistent with one or more embodiments of the present disclosure. FIG. 10B illustrates the calibration curve of SARS-CoV-2 in PBS, consistent with one or more embodiments of the present disclosure. Referring to FIGS. 10A-10B, glycoproteins of SARS-CoV-2 were correctly identified using exemplary label-free nanosensor and exhibited an electrochemical pattern with peak at voltage position of about −0.02 V and 0.05 V, which generated from interactions between the active functional groups of exemplary label-free nanosensor with electroactive hydrocarbon bonds of S spike glycoprotein of SARS-CoV-2. The unique peaks of SARS-CoV-2 appeared at voltage positions between −0.1 V and 0.1 V.

FIG. 10C illustrates a DPV pattern of SARS-CoV-2 in blood samples of infected people, consistent with one or more embodiments of the present disclosure. Referring to FIG. 10C, exemplary label-free nanosensor detected the unique peaks of the glycoprotein of SARS-CoV-2 in blood samples at voltage position between −0.1 V and 0.1 V and confirmed the existence of SARS-CoV-2 glycoprotein in the blood samples.

FIG. 10D illustrates a DPV pattern of SARS-CoV-2 in a saliva sample of an infected person, consistent with one or more embodiments of the present disclosure. Referring to FIG. 10D, exemplary label-free nanosensor also detected the lowest concentration or viral load of SARS-CoV-2 in the saliva sample of an infected person in the same voltage position between −0.1 V and 0.1 V compared with the blood samples.

FIG. 10E illustrates a DPV pattern of SARS-CoV-2 in an oropharyngeal swab sample of an infected person, consistent with one or more embodiments of the present disclosure. Referring to FIG. 10E, exemplary label-free nanosensor detected the trace of SARS-CoV-2 glycoproteins in the oropharyngeal swab of an infected person in the unique voltage position of SARS-CoV-2, between −0.1 V and 0.1 V.

Moreover, detection of SARS-CoV-2 using exemplary diagnostic kit was validated by comparing the results of 100 candidates who were known cases of positive and negative SARS-CoV-2 confirmed by RT-PCR as a clinical diagnostics standard. Comparative diagnostic results for detection of SARS-CoV-2 glycoproteins using exemplary diagnostic kit were presented in TABLE. 1. Among these 100 candidates, 60 and 40 were found to be positive and negative, respectively. In comparison with RT-PCR, exemplary diagnostic kit showed following results: TP: 57, FP: 16. TN: 24, and FN: 3 (TP: True Positive, FP: False Positive, TN: True Negative, and FN: False Negative)

TABLE 1
Comparative results of exemplary diagnostic kit and RT-PCR assay

| | | Percentage compared |
|---|---|---|
| Parameter | Formula | with RT-PCR (%) |
| Sensitivity | TP/(TP + FN) | 95 |
| Specificity | TN/(TN + FP) | 60 |
| Accuracy | (TP + TN)/(P + N) | 81 |
| False-negative rate | FN/P | 5 |
| False-positive rate | FP/N | 40 |

Referring to TABLE. 1, utilizing exemplary diagnostic kit showed an accuracy of about 81% and sensitivity of about 95% with respect to RT-PCR as the gold standard. It may result that the exemplary diagnostic kit, system, and method disclosed herein may be used as a power full assistant approach in a fast screening of different glycoproteins in patients who need a further medical examination.

### Example 5: Detection of New Castle Viruses Using Exemplary Diagnostic Kit

In this example, detection of glycoproteins of different strains of Newcastle disease virus (NDV) in different samples was done using exemplary diagnostic kit, similar to exemplary diagnostic kit 216. Evaluation of exemplary diagnostic kit performance was done by dropping about 100 μL of a biological sample containing NDV virus onto the top surface of the working electrode, the counter electrode, and the reference electrode. A voltage ranging from 0 V to 0.7 V was applied to exemplary diagnostic kit 216, and a DPV pattern of each sample was monitored following 30 seconds to 60 seconds.

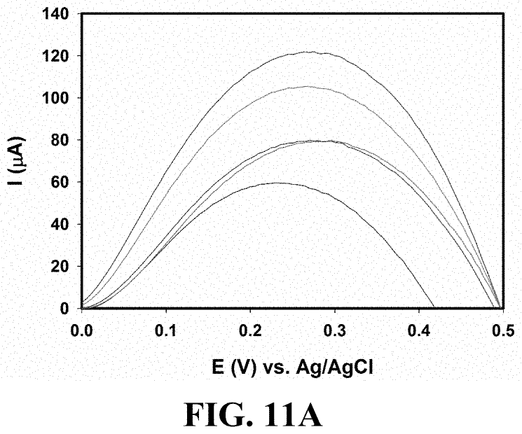

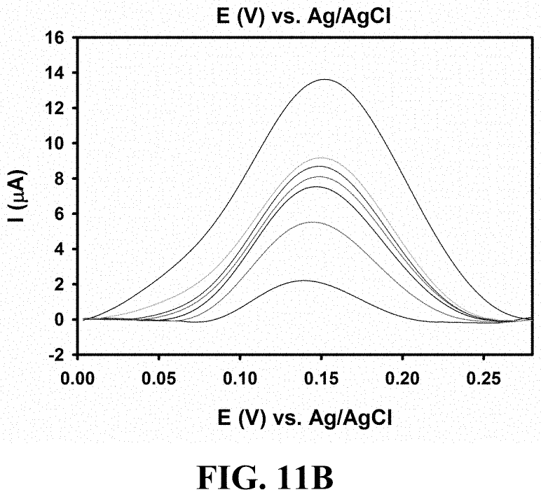

FIG. 11A illustrates a DPV pattern of Newcastle disease virus (LaSota strain consistent with one or more exemplary embodiments of the present disclosure. Referring to FIG. 11A, exemplary label-free nanosensor detected the LaSota strain of NDV at a voltage position of about 0.27 V. FIG. 11B illustrates a DPV pattern of Newcastle disease virus (V4 strain), consistent with one or more exemplary embodiments of the present disclosure. Referring to FIG. 11B, exemplary label-free nanosensor may detect the V4 strain of NDV at a voltage position of about 0.148 V. As a result, exemplary label-free nanosensor not only may detect diverse kinds of viral glycoproteins at different positions but also may distinguish various strains of a virus from each other.

### Example 6: Detection of Influenza Viruses Using Exemplary Diagnostic Kit

In this example, the detection of glycoproteins of different influenza virus strains was done using exemplary diagnostic kit, similar to exemplary diagnostic kit 216. Evaluation of the exemplary diagnostic kit performance was done by dropping about 100 μL of a biological sample, for instance, saliva, containing influenza virus onto the top surface of the working electrode, the counter electrode, and the reference electrode. A voltage ranging from 0 to 0.5 V was applied to exemplary diagnostic kit, and the DPV pattern of each sample was monitored following 30 seconds to 60 seconds.

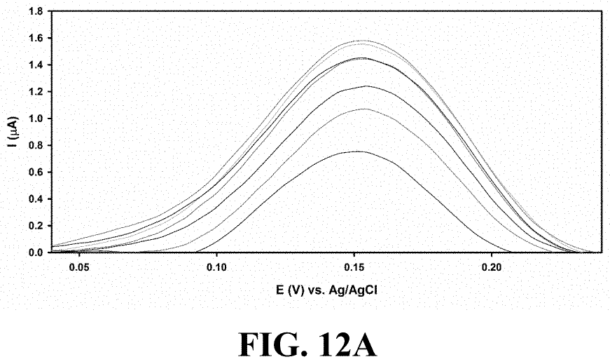

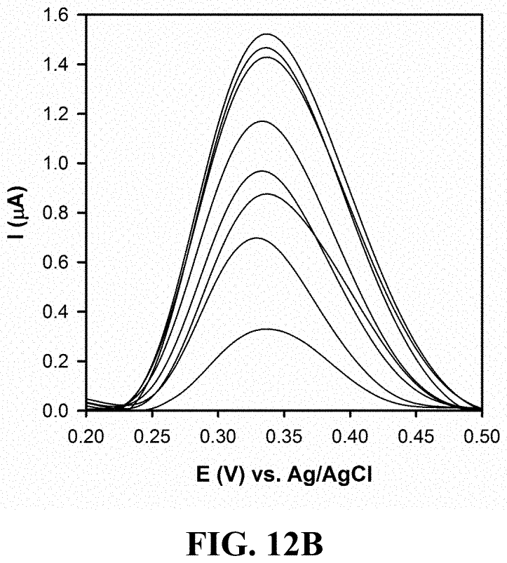

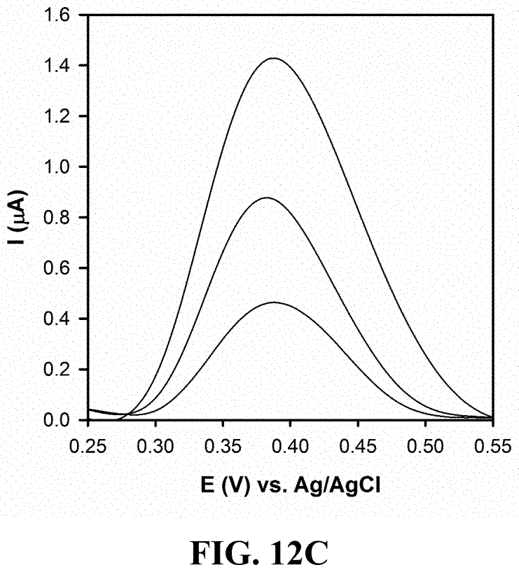

FIG. 12A illustrates a DPV pattern of avian influenza virus, consistent with one or more exemplary embodiments of the present disclosure. Referring to FIG. 12A, exemplary label-free nanosensor detected the unique electrochemical pattern of avian influenza at a voltage position of about 0.15 V, which is unique and different from the obtained standard electrochemical pattern of other viruses. As a result, exemplary label-free nanosensors showed a superior performance toward the detection of avian influenza viruses. FIG. 12B illustrates a DPV pattern of H₁N₁ strain of influenza virus, consistent with one or more exemplary embodiments of the present disclosure. Referring to FIG. 12B, exemplary label-free nanosensor detected the H₁N₁ strain of influenza virus at a voltage position of about 0.33 V, which is far different from the voltage position of avian influenza.

FIG. 12C illustrates a DPV pattern of H₃N₂ strain of influenza virus, consistent with one or more exemplary embodiments of the present disclosure. Referring to FIG. 12C, exemplary label-free nanosensor detected the H₃N₂ strain of influenza virus at a voltage position of about 0.38 V. As a result, exemplary label-free nanosensor 216 detected the trace of diverse kinds of influenza viruses at diverse voltage positions and distinguished the diverse kinds of influenza viruses via using a rapid electrochemical assay.

### Example 7: Detection of Collagens Using Exemplary Diagnostic Kit

In this example, the protein structure of diverse types of collagens was detected using exemplary diagnostic kit, similar to exemplary diagnostic kit 216. Evaluation of the exemplary diagnostic kit performance was done by dropping about 100 μL of a buffer sample containing collagen onto top surfaces of the working electrode, the counter electrode, and the reference electrode. A voltage ranging from −0.8 V to 0.8 V was applied to exemplary diagnostic kit, and the DPV pattern of each sample was monitored following 30 seconds to 60 seconds.

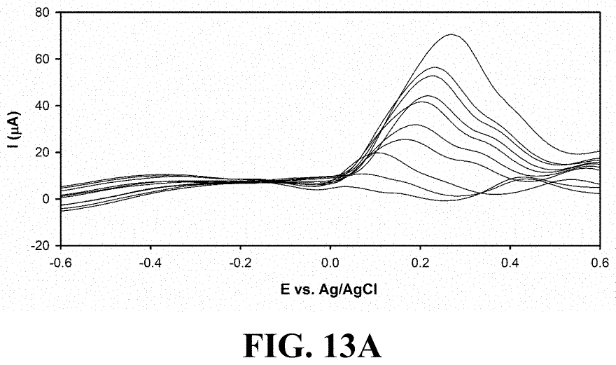

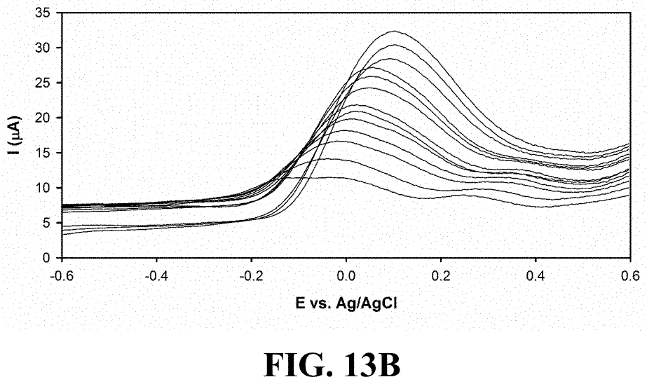

FIG. 13A illustrates a DPV pattern of human type I collagen, consistent with one or more exemplary embodiments of the present disclosure. Referring to FIG. 13A, human type I collagen exhibited a broad electrochemical pattern between voltage range of 0 V to 0.5 V that showed its unique voltage peak at a voltage position of about 0.23 V.

FIG. 13B illustrates a DPV pattern of porcine type I collagen, consistent with one or more exemplary embodiments of the present disclosure. Referring to FIG. 13B, exemplary label-free nanosensor may detect human type I collagen at voltage position of about 0.05 V with a broad electrochemical pattern from −0.2 V to 0.4 V. As a result, exemplary label-free nanosensor may be used for differentiable detection of collagens with diverse protein structure at apparently different voltage positions and with different electrochemical patterns.

### Example 8: Detection of Antibodies Using Exemplary Diagnostic Kit

In this example, detection of the protein structure of monoclonal IgG antibody of S1 part of S spike viral glycoprotein of SARS-CoV-2 was done using exemplary diagnostic kit, similar to exemplary diagnostic kit 216. Also, an electrochemical system similar to electrochemical system 300, including exemplary diagnostic kit 216, was utilized to process an exemplary method similar to method 100 for testing the presence of antibodies against SARS-CoV-2 in normal (not-infected with SARS-CoV-2) and infected cases with SARS-CoV-2.

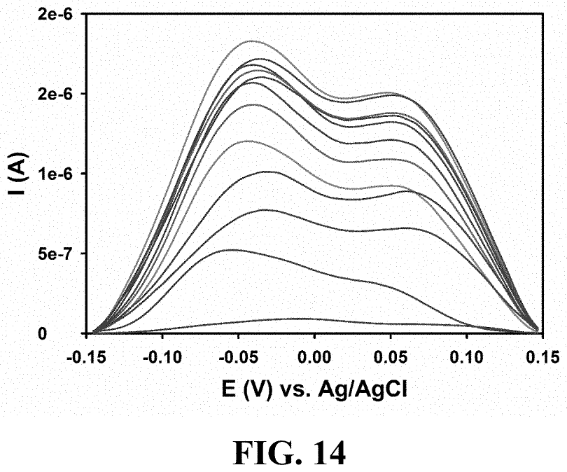

FIG. 14 illustrates a DPV pattern of monoclonal IgG antibody against S1 part of S spike glycoprotein of SARS-CoV-2, consistent with one or more exemplary embodiments of the present disclosure. Referring to FIG. 14, the electrochemical pattern of monoclonal IgG antibody of S1 part of S spike viral glycoprotein of SARS-CoV-2 included a peak at voltage positions between about −0.15 V and 0.15 V, which is generated due to interactions between the functional groups of exemplary label-free nanosensor with electroactive hydrocarbon bonds of monoclonal IgG antibody against S1 part of spike glycoprotein (S) of SARS-CoV-2. More importantly, the electrochemical pattern and voltage position of the monoclonal antibody of SARS-CoV-2 is nearly the same as its source antigen (SARS-CoV-2 antigen) due to similar active functional groups of monoclonal antibodies with and target antigen which was produced by the immune system toward specific targeting the viral antigen.

Moreover, detection of infected people with the infectious disease of COVID-19 using exemplary diagnostic kit 216 was validated by comparing the results of 40 candidates who were known cases of positive and negative COVID-19 confirmed by enzyme-linked immunosorbent assay (ELISA) as a clinical diagnostics standard. Comparative diagnostic results for detecting SARS-CoV-2 antibodies in blood samples using exemplary diagnostic kit were presented in TABLE. 2.

TABLE 2
Comparative results of exemplary diagnostic kit and ELISA assay as a
gold standard with a cutoff point of 0.2 μA

| | | Percentage compared |
|---|---|---|
| Parameter | Formula | with RT-PCR (%) |
| Sensitivity | TP/TP + FN | 100 |
| Specificity | TN/TN + FP | 85 |
| Negative prediction value | TN/TN + FN | 100 |
| Positive prediction value | TP/TP + FP | 86.95 |
| False-negative rate | FN/FN + TP | 0 |
| False-positive rate | FP/FP + TN | 15 |
| False discovery rate | FP/FP + TP | 13.04 |
| Accuracy | (TP + TN)/P + N | 92.5 |
| False-negative rate | FN/P | 0 |
| False-positive rate | FP/N | 15 |

Referring to TABLE. 2 among these 40 candidates, 20 and 20 were found to be positive and negative, respectively. In comparison with RT-PCR, exemplary diagnostic kit showed following results: TP: 20, FP: 3, TN: 17, and FN: 0 (TP: True Positive, FP: False Positive, TN: True Negative. and FN: False Negative). As a result, exemplary diagnostic kit showed 100% sensitivity and 85% specificity for detecting antibodies against spike glycoprotein of SARS-CoV-2.

### Example 9: Detection of SARS-CoV-2 Antigen Using Cyclodextrin Modified go Sheets Along with Gold and Silver Nanoparticles as Amplifying Agents

In this example, detection of glycoproteins of SARS-CoV-2 in buffer samples was done using exemplary diagnostic kit, similar to exemplary diagnostic kit 216. Also, an electrochemical system similar to electrochemical system 300, including exemplary diagnostic kit 216, was utilized to process an exemplary method similar to method 100 for testing the presence of SARS-CoV-2 glycoproteins in a buffer solution. Exemplary diagnostic kit 216 used in this example included a working electrode, including modified GO sheets and amplifying agents loaded onto the modified GO sheets. The modified GO sheets included GO sheets modified with EDC, NHS, 8H, hydroxylammonium chloride, and β-cyclodextrin. Additionally, silver nanowires (Ag NW) and gold nanostars (Au NS) were selected as amplifying agents and used along with the modified GO sheets to improve the intensity of the response of exemplary label-free nanosensor to the glycoproteins of SARS-CoV-2 in a buffer solution.

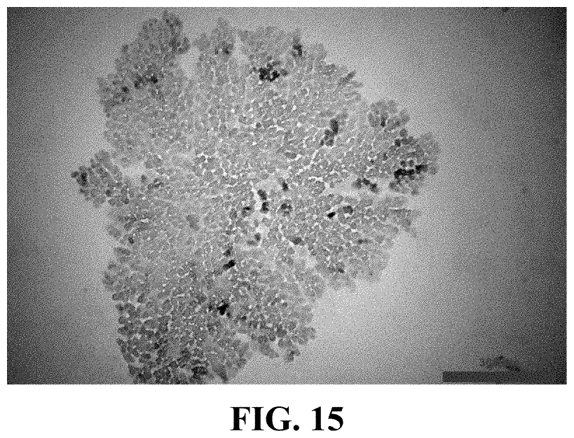

FIG. 15 illustrates the transmission electron microscopy (TEM image of modified GO sheets, including GO sheets modified with (EDC) NHS, 8H, hydroxylammonium chloride β-cyclodextrin, consistent with one or more embodiments of the present disclosure. Referring to FIG. 15, modification of GO-8H-EDC-NHS with β-cyclodextrin significantly changed the surface morphology of modified GO sheets and provided too many porous active sites for interaction with active functional groups of SARS-CoV-2 glycoproteins in aqueous samples.

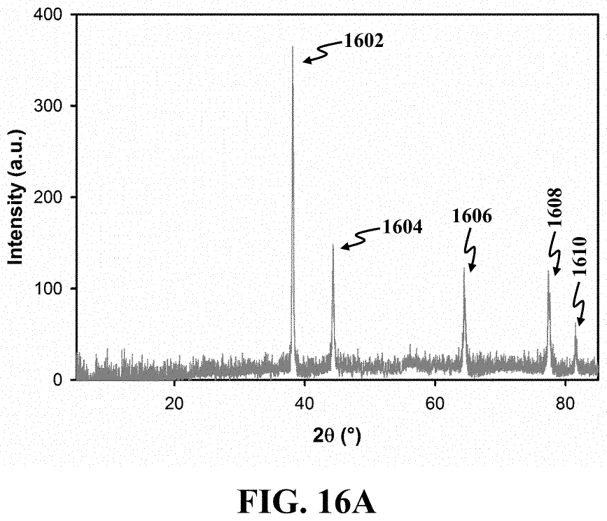

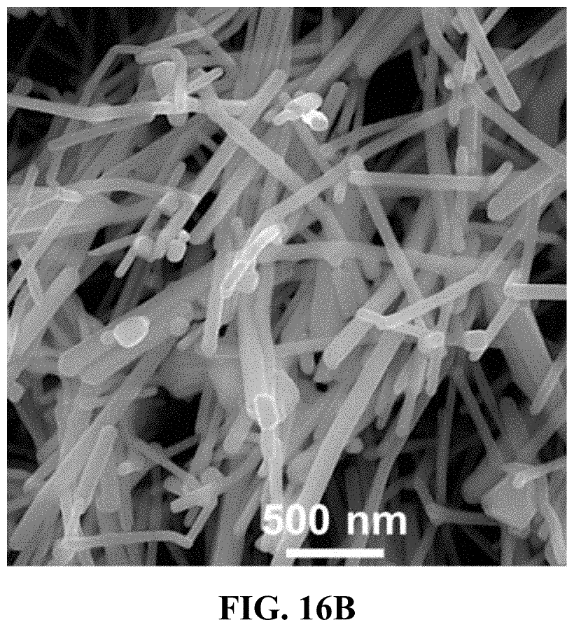

FIG. 16A illustrates an X-ray powder diffraction (XRD) spectrum of silver nanowires (Ag NWs), consistent with one or more embodiments of the present disclosure. Referring to FIG. 16A, Ag NWs were successfully produced and exhibited standard crystalline planes of Ag₄ compound with cubic crystal system including (111) 1602, (002) 1604, (022) 1606, (113) 1608, and (222) 1610, which is in accord with reference 96-901-1608. FIG. 16B illustrates field-emission scanning electron microscopy (FESEM) image of Ag NWs, consistent with one or more exemplary embodiments of the present disclosure. Referring to FIG. 16B, the FESEM image of Ag NWs showed well-resolved nanowire morphology, which furtherly confirms the successful synthesis of Ag NWs.

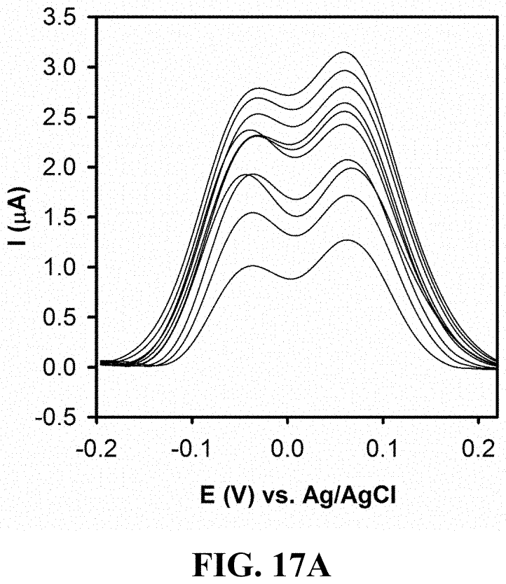

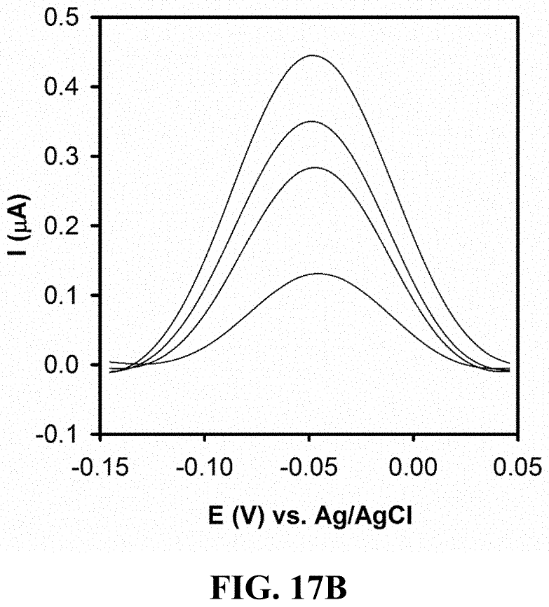

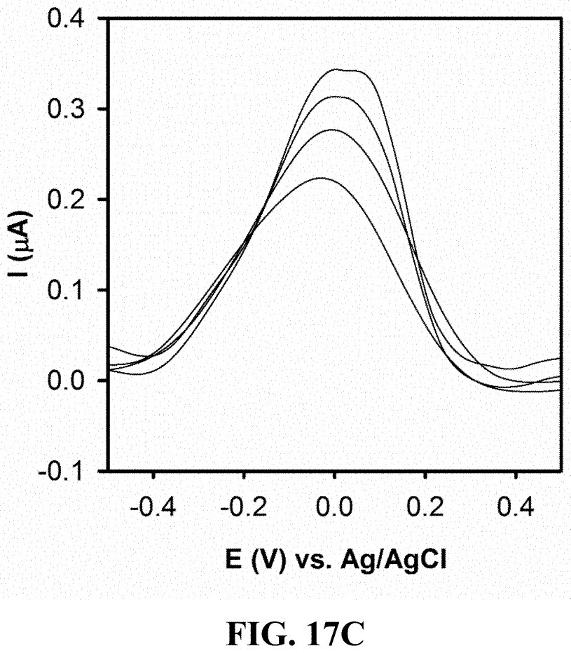

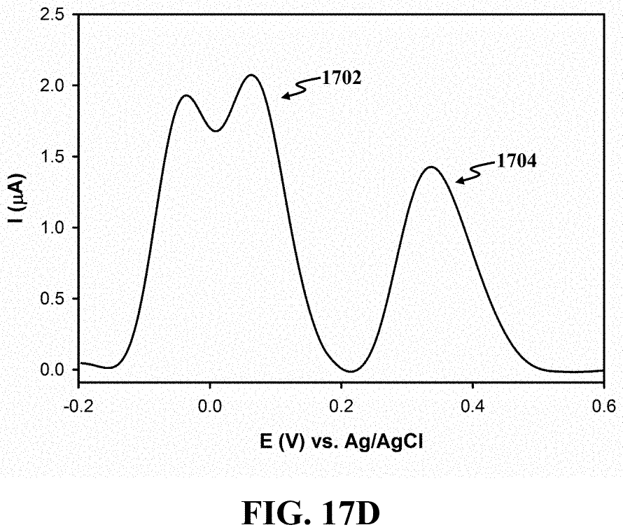

FIG. 17A illustrates a DPV pattern of modified GO sheets, including GO sheets modified with EDC, NHS, 8H, hydroxylammonium chloride, and β-cyclodextrin, consistent with one or more exemplary embodiments of the present disclosure. Referring to FIG. 17A, the DPV pattern of GO sheets modified with EDC, NHS, 8H, hydroxylammonium chloride, and β-cyclodextrin showed a similar DPV pattern to the modified GO sheets of Example 4, which were modified with EDC, NHS, 8H, hydroxylammonium chloride. The DPV pattern also showed an electrochemical pattern at voltage positions between −0.2 V and 0.2 V with twin peaks at −0.03 and 0.06 V. Also, further modification of modified GO sheets of Example 4 with β-cyclodextrin leads to improved quality of obtained electrochemical patterns and electrochemical peaks of SARS-CoV-2 antigen in buffer solution.

FIG. 178 illustrates a DPV pattern of SARS-CoV-2 glycoproteins utilizing exemplary label-free nanosensor including modified GO sheets, containing GO sheets modified with EDC, NHS, 8H, hydroxylammonium chloride, and β-cyclodextrin, along with Ag NWs as an amplifying agent, consistent with one or more embodiments of the present disclosure. Referring to FIG. 17B, integration of modified GO sheets with Ag NWs significantly increased the current response of the obtained electrochemical patterns with improved intensity, which is favorable for detecting the lowest concentration of viral glycoproteins in biological samples. Accordingly, integration of modified GO sheets with Ag NWs changed the wide electrochemical pattern of SARS-CoV-2 glycoproteins to a single peak electrochemical pattern with a domain from −0.15 V to 0.05 V and a single peak at a voltage position of −0.04 V.

FIG. 17C illustrates a DPV pattern of SARS-CoV-2 glycoproteins obtained by utilizing exemplary label-free nanosensor containing modified GO sheets, including GO sheets modified with EDC, NHS, 8H, hydroxylammonium chloride, and β-cyclodextrin, along with Au NSs as an amplifying agent, consistent with one or more embodiments of the present disclosure. Referring to FIG. 17C, integration of Au NSs with modified GO sheets also considerably improved the response of exemplary label-free nanosensor to viral glycoproteins of SARS-CoV-2 glycoproteins. Similar to Ag NWs, the Au NSs also improved the response of exemplary label-free nanosensor to viral glycoproteins of SARS-CoV-2 glycoproteins; however, the Au NSs extended the domain of electrochemical pattern of SARS-CoV-2 glycoproteins to voltage positions between −0.4 V and 04 V with a single peak at voltage position of about 0.01 V.

Furthermore, exemplary label-free nanosensors may also be capable of simultaneously detecting diverse kinds of pathogenic viruses in biological/non-biological media. FIG. 17D illustrates a DPV pattern of glycoproteins of SARS-CoV-2 and H₁N₁ strain of influenza virus detected utilizing an exemplary label-free nanosensor containing modified GO sheets, including GO sheets modified with EDC, NHS, 8H, hydroxylammonium chloride, and β-cyclodextrin, consistent with one or more embodiments of the present disclosure. Referring to FIG. 17D, the modified GO sheet has simultaneously detected the glycoproteins of SARS-CoV-2 1702 and H₁N₁ influenza virus 1704 within a biological sample. As a result, exemplary label-free nanosensors have a capability for differentiable or simultaneous detection of pathogenic viruses within biological/non-biological media.

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such away. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second, and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure and is not to be interpreted as reflecting an intention that the claimed implementations require more features than are expressly recited in each claim. Rather, as the following claims reflect, the inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in the light of the attached claims and their equivalents. Also, various modifications and changes may be made within the scope of the attached claims.

What is claimed is:

1. A method for detecting glycoproteins in aqueous samples, the method comprising:
putting an aqueous sample in contact with a diagnostic kit, the diagnostic kit comprising:
a working electrode comprising a label-free nanosensor deposited on a substrate, the label-free nanosensor comprising:
a modified graphene oxide (GO) sheet comprising a modifying agent conjugated to a GO sheet, the modifying agent comprising 1-ethyl-3-3-dimethylaminopropyl) carbodiimide (EDC)·N-hydroxysuccinimide (NHS), 8-hydroxyquinoline (8H), and hydroxylammonium chloride; and
a signal amplifying agent loaded onto the modified GO sheet, the signal amplifying agent comprising at least one of an anine-functionized gold nanoparticle and a silver nanoparticle;
a counter electrode; and
a reference electrode;
obtaining an electrochemical pattern of the aqueous sample by applying an electrical potential to the diagnostic kit; and
detecting a glycoprotein status of the aqueous sample based on presence of a peak in the electrochemical pattern of the aqueous sample, comprising:
detecting that a glycoprotein is present in the aqueous sample if the electrochemical pattern contains the peak, the peak comprising a current intensity and a voltage position; and
detecting that a glycoprotein is absent in the aqueous sample if the electrochemical pattern lacks the peak.

2. The method of claim 1 further comprising identifying the glycoprotein in the aqueous sample by comparing the peak of the electrochemical pattern with standard peaks of standard electrochemical patterns in a database, the database comprising a plurality of datasets, each dataset associated with a standard glycoprotein, each dataset comprising:
a standard electrochemical pattern of the standard glycoprotein comprising a standard peak, the standard peak comprising:
a standard voltage position; and
a standard current intensity; and
a calibration curve relating the standard current intensity of the standard elect chemical pattern to a concentration of the standard glycoprotein.

3. The method of claim 2, wherein comparing the peak of the electrochemical pattern with the standard peaks of the standard electrochemical patterns in the database, comprises:
determining a type of the glycoprotein by finding a standard glycoprotein in the database through comparing the voltage position of the peak with standard voltage positions of the standard peaks in the database; and
measuring a concentration of the glycoprotein based on the calibration curve of the standard glycoprotein.

4. The method of claim 1 further comprising generating a database, generating the database comprising:
obtaining a plurality of standard electrochemical patterns of a plurality of standard glycoproteins, each standard electrochemical pattern of the standard glycoprotein comprising a standard peak, the standard peak comprising:
a standard voltage position; and
a standard current intensity; and
plotting a calibration curve for each standard glycoprotein by relating the standard current intensity of each standard electrochemical pattern to a concentration of the standard glycoprotein.

5. The method of claim 1, wherein applying the electrical potential to the diagnostic kit comprises applying a predetermined electrical potential between −1 V and 1 V to the diagnostic kit.

6. The method of claim 1, wherein obtaining the electrochemical pattern of the aqueous sample comprises obtaining at least one of a cyclic voltammetry (CV) pattern, a differential pulse voltammetry (DPV) pattern, an electrochemical impedance spectroscopy (EIS) pattern, a square wave voltammetry (SWV) pattern, and a pattern of an amperometry assay of the aqueous sample.

7. The method of claim 1, wherein applying the electrical potential to the diagnostic kit comprises applying a predetermined electrical potential to the diagnostic kit through an electrochemical system connected to the diagnostic kit.

8. The method of claim 1, wherein detecting glycoproteins in the aqueous samples comprises detecting at least one of viral glycoproteins, collagens, and antibodies in the aqueous samples.

9. The method of claim 7, wherein detecting the viral glycoproteins comprises detecting at least one of coronaviruses, influenza viruses, and Newcastle disease viruses.

10. The method of claim 1, wherein the modifying agent comprises the EDC with a concentration between 1% and 20% by weight of the GO sheet, the NHS with a concentration between 1% and 20% by weight of the GO sheet, the 8H with a concentration between 10% and 50% by weight of the GO sheet, and the hydroxylammonium chloride with a concentration between 10% and 50% by weight of the GO sheet.

11. The method of claim 1, wherein the modifying agent further comprises cyclodextrin with a concentration between 10% and 50% by weight of the GO sheet.

12. The method of claim 1, wherein the amine-functionalized gold nanoparticle comprises at least one of an amine-functionalized gold nanostar, an amine-functionalized gold nanorod, an amine-functionalized gold nanowire, an amine-functionalized gold spherical nanoparticle, an amine-functionalized gold nanoplate, and an amine-functionalized gold cubic nanostructure.

13. The method of claim 1, wherein putting the aqueous sample in contact with the diagnostic kit comprises putting at least one of a serum sample, a urine sample, a cerebrospinal fluid sample, a saliva sample, a blood sample, a mucus sample, a swab sample, and a buffer sample in contact with the diagnostic kit.

14. A diagnostic kit for detecting glycoproteins in aqueous samples, comprising:
a working electrode comprising a label-free nanosensor deposited on a substrate, the label-free nanosensor comprising:
a modified graphene oxide (GO) sheet comprising a modifying agent conjugated to a GO sheet, the modifying agent comprising 1-ethyl-3-(3-dimethylaminopropyl) carbodiimide (EDC), N-hydroxysuccinimide (NHS), 8-hydroxyquinoline (8H), and hydroxylammonium chloride; and
a signal amplifying agent loaded onto the modified GA sheet, the signal amplifying agent comprising at least one of an amine-functionalized gold nanoparticle and a silver nanoparticle;
a counter electrode; and
a reference electrode.

15. The electrochemical device of claim 14, wherein the modifying agent comprises the EDC with a concentration between 1% and 20% by weight of the GO sheet, the NHS with a concentration between 1% and 20% by weight of the GO sheet, the 8H with a concentration between 10% and 50% by weight of the GO sheet, and the hydroxylammonium chloride with a concentration between 10% and 50% by weight of the GO sheet.

16. The electrochemical device of claim 14, wherein the modifying agent further comprises cyclodextrin with a concentration between 10% and 50% by weight of the GO sheet.

17. The electrochemical device of claim 14, wherein the amine-functionalized gold nanoparticle comprises at least one of an amine-functionalized gold nanostar, an amine-functionalized gold nanorod, an amine-functionalized gold nanowire, an amine-functionalized gold spherical nanoparticle, an amine-functionalized gold nanoplate, and an amine-functionalized gold cubic nanostructure.

18. The electrochemical device of claim 14, wherein the glycoproteins comprises at least one of viral glycoproteins, collagens, and antibodies.

19. The electrochemical device of claim 18, wherein the viral glycoproteins comprises glycoprotins of at least one of coronaviruses, influenza viruses, and Newcastle disease virus.

20. The electrochemical device of claim 11, wherein the aqueous sample comprises at least one of a serum sample, a urine sample, a cerebrospinal fluid sample, a saliva sample, a blood sample, a mucus sample, a swab sample, and a buffer sample.
